(19) United States
(12) Patent Application Publication
(10) Pub. No.: US 2024/0207361 A1
(43) Pub. Date: Jun. 27, 2024

(54) ERYTHROPOIETIN-DERIVED PEPTIDES FOR TREATING RELAPSING-REMITTING MULTIPLE SCLEROSIS

(71) Applicant: The United States Government as represented by the Department of Veterans Affairs, Washington, DC (US)

(72) Inventors: Peter C. Dowling, Sea Girt, NJ (US); Wei Lu, Sea Girt, NJ (US)

(21) Appl. No.: 18/555,169

(22) PCT Filed: Apr. 14, 2022
(86) PCT No.: PCT/US2022/024785

§ 371 Date: Oct. 12, 2023

Related U.S. Application Data
(60) Provisional application No. 63/175,742, filed on Apr. 16, 2021.

(51) Int. Cl. A61K 38/18; A61P 25/28; A61P 37/06

(52) U.S. Cl. CPC A61K 38/1816; A61P 25/28; A61P 37/06

(57) ABSTRACT

Described herein are dosing regimens and methods of treating multiple sclerosis with an effective amount of an erythropoietin (EPO)-derived peptide to provide sustained therapeutic effects after withdrawal of the EPO-derived peptide. The dosing regimens and methods include a treatment cycle followed by a rest phase, wherein the EPO-derived peptide is not administered during the rest phase.

### CROSS REFERENCE TO RELATED APPLICATIONS

This application claims the benefit of U.S. Provisional Application No. 63/175,742, filed Apr. 16, 2021. The content of this earlier filed application is hereby incorporated by reference herein in its entirety.

### STATEMENT REGARDING FEDERALLY FUNDED RESEARCH

This is invention was made with government support under grant number TX001305 awarded by the Department of Veterans Affairs and under grant number NS073526 awarded by the National Institutes of Health. The government has certain rights in the invention.

### INCORPORATION OF THE SEQUENCE LISTING

The present application contains a sequence listing that is submitted via EFS-Web concurrent with the filing of this application, containing the file name “37759_0378 P1_Sequence_Listing.txt” which is 12,288 bytes in size, created on Mar. 24, 2022, and is herein incorporated by reference in its entirety.

### BACKGROUND

Potent beneficial immunomodulatory and anti-inflammatory effects of whole molecule erythropoietin (EPO) have been demonstrated in a variety of animal disease models including experimental autoimmune encephalomyelitis (EAE); however, excessive hematopoiesis limits its use in clinical applications. This animal model has been used widely by many investigators to study disease pathogenesis and to explore new therapies for its human counterpart, multiple sclerosis (MS). MS a disorder of unknown cause, and is defined clinically by characteristic symptoms, signs and progression, and is defined pathologically by scattered areas of inflammation and demyelination affecting the brain, optic nerves and spinal cord white matter. It is widely believed that the pathogenesis of MS involves an immune mediated inflammatory demyelinating process. While no cure for MS exists, new disease modifying therapies are needed.

### SUMMARY

Disclosed herein are dosing regimens comprising at least one treatment cycle followed by a rest phase, wherein the treatment cycle comprises administering an effective amount of an erythropoietin (EPO)-derived peptide to allow for a sustained therapeutic effect after withdrawal of the EPO-derived peptide, wherein the EPO-derived peptide is not administered during the rest phase.

Disclosed herein are methods of treating relapsing-remitting multiple sclerosis, the methods comprising administering to a subject an effective amount of an erythropoietin (EPO)-derived peptide for at least one treatment cycle, wherein the treatment cycle comprises an effective amount of the EPO-derived peptide to allow for a sustained therapeutic effect after withdrawal of the EPO-derived peptide, wherein the treatment cycle is followed by a rest phase, and wherein the EPO-derived peptide is not administered during the rest phase.

Disclosed herein are dosing regimens comprising at least one treatment cycle followed by a rest phase, wherein the treatment cycle comprises administering an effective amount of an erythropoietin (EPO)-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the EPO-derived peptide is not administered during the rest phase.

Disclosed herein are methods of treating relapsing-remitting multiple sclerosis, the methods comprising administering to a subject an effective amount of an erythropoietin (EPO)-derived peptide for at least one treatment cycle, wherein the treatment cycle comprises administering an effective amount of the EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the treatment cycle is followed by a rest phase, wherein the EPO-derived peptide is not administered during the rest phase.

Disclosed herein are methods of reducing A1 astrocyte activation in spinal cord, the methods comprising administering to a subject an effective amount of an erythropoietin (EPO)-derived peptide for at least one treatment cycle, wherein the treatment cycle comprises administering an effective amount of the EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the treatment cycle is followed by a rest phase, wherein the EPO-derived peptide is not administered during the rest phase.

Disclosed herein are methods of decreasing complement component C3, the methods comprising administering to a subject an effective amount of an erythropoietin (EPO)-derived peptide for at least one treatment cycle, wherein the treatment cycle comprises administering an effective amount of the EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the treatment cycle is followed by a rest phase, wherein the EPO-derived peptide is not administered during the rest phase.

Disclosed herein are methods of treating a disease, disorder or condition having an inflammatory or autoimmune component in a subject in need thereof, the methods comprising administering to a subject an effective amount of an erythropoietin (EPO)-derived peptide for at least one treatment cycle, wherein the treatment cycle comprises administering an effective amount of the EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the treatment cycle is followed by a rest phase, wherein the EPO-derived peptide is not administered during the rest phase, wherein the composition is effective at ameliorating at least one symptom from at least one disease, disorder, or condition having an inflammatory or autoimmune component.

Disclosed herein is the use of an erythropoietin (EPO)-derived peptide for the production of a medicament for the treatment of multiple sclerosis (MS) in a patient wherein the EPO-derived peptide consists of the amino acid sequence GCAEHCSLNENITVPDTKV (SEQ ID NO: 1).

Disclosed herein is the use of an erythropoietin (EPO)-derived peptide for the production of a medicament for the treatment of multiple sclerosis (MS) in a patient, the treatment comprising a first treatment cycle of the EPO-derived peptide followed by at least one further treatment cycle of the EPO-derived peptide, in which each treatment cycle comprises 1-14 doses which are applied on consecutive days, wherein the daily dose is >0) and ≤ 10 mg, and wherein each treatment cycle is separated from the next treatment cycle by at least 1-24 months.

Other features and advantages of the present compositions and methods are illustrated in the description below, the drawings, and the claims.

### BRIEF DESCRIPTION OF THE DRAWINGS

FIG. 1 shows the relative expression of GFAP mRNA and luciferase in GFAP-Luc/SJL EAE mice. mRNA was extracted from SJL/J EAE mice brains at days 0, 7 and 14 post immunization and quantified using real time PCR. A strong correlation was noted between GFAP mRNA and luciferase. SJL/J EAE mice showed increased expression of GFAP and luciferase on days 7 and 14 (n=3, 6-7-fold increase at day 7: 23-25 fold increase at day 14).

FIG. 2 shows the clinical scores in JM-4 treated monophasic MOG EAE mice. MOG immunized GFAP-Luc/C57 mice developed significant neurologic impairment on 11 days post immunization. Treatment with JM-4 (5 μg IV daily for 12 days) was initiated on day 9. Peak clinical scores in JM-4 treated MOG animals were significantly lower compared to sham treated MOG EAE mice (n=8 JM-4 treated, n=8 sham treated, p<0).05).

FIGS. 3A-C show a marked positive treatment effect with JM4 was also seen by GFAP-luc BLI assessment that correlated with clinical scores. FIG. 3A shows in-vivo imaging following treatment with JM-4 in MOG-induced EAE. Sham-treated GFAP-Luc/C57 mice (upper panel) and JM-4 treated mice with MOG-induced EAE (lower panel) were monitored using BLI over 21 days. Treatment with JM-4 (5 μg IV daily for 12 days) was started on day 9. This JM-4 treated animal exhibited lower spinal cord peak intensity within two days after treatment and virtual absence of spinal cord signal by day 4 (FIGS. 3B, 3C). Relative intensity of bioluminescence in forebrain (FIG. 3B) and spinal cord (FIG. 3C) in GFAP-Luc/C57 MOG-induced EAE mice. FIGS. 3B-C show peak bioluminescent values were significantly lower in JM-4 treated GFAP-Luc/C57 EAE mice in both forebrain and spinal cord (brain n=8, p<0).05: spinal cord n=8, p<0.05). GFAP-Luc/C57 MOG-induced EAE mice were treated with JM-4 (5 μg IV daily for 12 days) starting on day 9. Relative intensity was measured as the ratio of photon intensity in the lesion compared to the left ear value.

FIGS. 4A-B show longitudinal assessment of BLI and clinical scores in two untreated GFAP-Luc/SJL relapsing-remitting EAE mice. Black bars represent bioluminescent flare ups. FIG. 4A shows that following initial clinical presentation (days 14-22), the mouse remained asymptomatic from day 28 to 73 (upper panel). Despite strong BLI enhancement there was no clinical correlate (days 54-64). In contrast, the third episode of BLI enhancement corresponded to a strong clinical relapse with severe paralysis (days 78-81). FIG. 4B shows that in the second animal (lower panel), a correlation was consistently seen between BLI and clinical scoring. Increased BLI signal in the brain and spinal cord was associated with increased clinical deficit in three distinct episodes (days 11-30, day 45-60, days 90-100).

FIG. 5 shows clinical scores in long term PLP-induced relapsing-remitting EAE. GFAP-luc/SJL mice were treated for 12 days with JM-4 (5 μg IV) starting on day 9. Average clinical scores in the JM-4 treated group remained remarkably reduced compared to the sham-treated group for over five months (n=4 JM-4 treated, n=5 sham treated, p<0.05 at day 60 onward).

FIG. 6 shows the positive treatment effect on flare ups of spinal cord bioluminescence in GFAP-Luc/SJL relapsing-remitting EAE mice treated with JM-4 (5 μg IV daily for 12 days). Episodes of spinal cord bioluminescent signal over background during disease course were calculated. JM-4 treatment significantly decreased the number of imaging flare ups compared to flare ups in the sham-treated group (n=8 JM-4 treated, n=7 sham treated, p<0.05). Grey bars represent standard error (mean+/−SEM sham treated 10.29+/−2.447, JM-4 treated 4.0+/−1.018).

FIGS. 7A-B shows that JM-4 is protective against spinal cord demyelination and axonal injury in SJL/J relapsing-remitting EAE mice. FIG. 7A shows luxol fast blue stain for myelin (left sided panels) in sham treated EAE mice shows pronounced demyelination and vacuolization in the ventral white matter of the spinal cord compared to JM-4 treated animals. SMI-32-stained axons (right sided panels) in JM-4 treated mice show a large reduction in number of injured axons rimming the periphery of the ventral spinal cord (white matter) compared to sham treated EAE control mice. FIG. 7B shows that JM4 therapy attenuates blood-brain barrier breakdown. Spinal cord sections were stained for mouse IgG to determine if serum leakage occurred. In a normal mouse spinal cord section, no IgG is detected. In contrast, saline treated EAE mice showed increased immunoreactivity most notably seen in the white matter of the spinal cord. Treatment with JM4 profoundly reduced the amount of IgG immunoreactivity in EAE cord back to close to normal.

FIGS. 8A-B shows treatment with JM-4 leads to significantly reduced A1 astrocyte activation in the spinal cord of EAE mice. FIG. 8A shows the quantitative analysis showing significant up regulation of complement component C3 in EAE mice compared to normal mice, and significant decrease in C3 immunoreactivity following treatment with JM-4 in EAE mice (data represent mean+/−s.e.m). FIG. 8B shows immunofluorescence images showing reduction in both GFAP (green) and C3 (red) immunoreactivity in the spinal cord of JM-4 treated EAE mice. Merged images showed most C3 immunoreactivity is found on in GFAP positive astrocytes (yellow) (n=7 per group, p<0.05, ANOVA followed by Bonferroni comparison between EAE mice and JM-4 treated EAE mice).

### DETAILED DESCRIPTION

Many modifications and other embodiments of the present disclosure set forth herein will come to mind to one skilled in the art to which this disclosure pertains having the benefit of the teachings presented in the foregoing descriptions and the associated drawings. Therefore, it is to be understood that the present disclosure is not to be limited to the specific embodiments disclosed and that modifications and other embodiments are intended to be included within the scope of the appended claims. Although specific terms are employed herein, they are used in a generic and descriptive sense only and not for purposes of limitation.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present disclosure, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow; plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

As used in the specification and in the claims, the term “comprising” can include the aspects “consisting of” and “consisting essentially of.” “Comprising” can also mean “including but not limited to.”

As used in the specification and the appended claims, the singular forms “a,” “an” and “the” can include plural referents unless the context clearly dictates otherwise. Thus, for example, reference to “a compound” includes mixtures of compounds: reference to “a pharmaceutical carrier” includes mixtures of two or more such carriers, and the like. The word “or” as used herein means any one member of a particular list and also includes any combination of members of that list.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject: a cell (either within a subject, taken directly from a subject, or a cell maintained in culture or from a cultured cell line): a cell lysate (or lysate fraction) or cell extract: or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and new born subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for multiple sclerosis, such as, for example, prior to the administering step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. Treatment can also be administered to a subject to ameliorate one more signs of symptoms of a disease, disorder, and/or condition. For example, the disease, disorder, and/or condition can be relating to multiple sclerosis.

The term “adjuvant” as used herein refers to any component which improves the characteristics, efficacy or potency of a formulation, drug, or immunological agent.

The term “administer” as used herein refers to dispensing, supplying, applying, giving, apportioning or contributing. The terms “administering” or “administration” are used interchangeably and include in vivo administration, as well as administration directly to tissue ex vivo. Generally, compositions may be administered systemically either orally, buccally, parenterally, topically, by inhalation or insufftation (i.e., through the mouth or through the nose), or rectally in dosage unit formulations containing the conventional nontoxic pharmaceutically acceptable carriers, adjuvants, and vehicles as desired, or may be locally administered by means such as, but not limited to, injection, implantation, grafting, topical application, or parenterally. The term “parenteral” as used herein refers to introduction into the body by way of an injection (i.e., administration by injection), including, for example, subcutaneously (i.e., an injection beneath the skin), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents.

Additional administration may be performed, for example, intravenously, pericardially, orally, via implant, transmucosally, transdermally, intramuscularly, subcutaneously, intraperitoneally, intrathecally, intralymphatically, intralesionally, or epidurally. Administration can be performed, for example, once, a plurality of times, and/or over one or more extended periods.

The term “carrier” as used herein refers to an organic or inorganic ingredient, natural or synthetic, with which the active ingredient is combined to facilitate the application that does not cause significant irritation to an organism and does not abrogate the biological activity and properties of the composition of the described invention. Carriers must be of sufficiently high purity and of sufficiently low toxicity to render them suitable for administration to a subject being treated. The carrier can be inert, or it can possess pharmaceutical benefits, cosmetic benefits or both.

The term “contact” as used herein refers to a state or condition of touching or of immediate or local proximity. The term “contacting” as used herein refers to bringing or putting in contact. Contacting a composition to a target destination, such as, but not limited to, an organ, tissue, cell, or tumor, may occur by any means of administration known to the skilled artisan.

The term “EPO-derived oligopeptide”, “erythropoietin (EPO)-derived oligopeptide”, “EPO AB loop peptide”, and “short EPO peptide” are used interchangeably to refer to an isolated or synthetic peptide encoding a fragment of mammalian erythropoietin (EPO). The term “oligopeptide” as used herein refers to any molecule that contains a small number (for example, 2 to about 30) of amino acid residues connected by peptide bonds. The term “EPO derived oligopeptide” as used herein also includes an isolated or synthetic peptide encoding a fragment of mammalian erythropoietin (EPO), which contains additional chemical moieties, which are not normally a part of the peptide.

“Dosing regimen” as used herein refers to at least one treatment cycle followed by at least one rest phase. A dosing regimen can include more than one treatment cycle and more than one rest phase. For example, a dosing regimen can be a one week treatment cycle followed by a 5 month rest phase. Another example can be a two week treatment cycle followed by a one year rest phase and then a one week treatment cycle followed by a one year rest phase.

As used herein, the term “treatment cycle” refers to the administration of EPO-derived peptide for an established period of time. A treatment cycle includes a wide range of dosages of EPO-derived peptides as well as different lengths of time for administering the EPO-derived peptides. For example, a treatment cycle can be a three month period wherein an EPO-derived peptide can be administered twice a week for the three month period.

“Dose” or “dosage” as used herein refers to a specific quantity of a therapeutic agent, such as an EPO-derived peptide, that is taken at specific times.

As used herein, “effective amount” is meant to mean a sufficient amount of the composition or EPO-derived peptide to provide the desired effect. For example, an effective amount of an EPO-derived peptide can be an amount that provides a therapeutic affect and provides sustained therapeutic effects after withdrawal of the treatment. An effective amount of an EPO-derived peptide is an amount that is able to cause a benefit illustrated by decreasing relapsing-remitting multiple sclerosis, reducing A1 astrocyte activation in spinal cord, decreasing complement component C3, and/or ameliorating at least one symptom from at least one disease, disorder, or condition having an inflammatory or autoimmune component, as well as an amount that allows for a sustained therapeutic effect after withdrawal of the EPO-derived peptide. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

As used herein, “sustained therapeutic effect” is a therapeutic effect that persists after the therapeutic has been withdrawn.

“Rest phase” as used herein refers to a period of time wherein an EPO-derived peptide is not administered.

“Peptide” as used herein refers to any peptide, oligopeptide, polypeptide, gene product, expression product, or protein. A peptide is comprised of consecutive amino acids. The term “peptide” encompasses naturally occurring or synthetic molecules.

As used herein, “treat” is meant to mean administer one of the disclosed compositions to a subject, such as a human or other mammal (for example, an animal model), that has multiple sclerosis, in order to prevent or delay a worsening of the effects of the disease or condition, or to partially or fully reverse the effects of the disease.

As used herein, “prevent” is meant to mean minimize the chance that a subject who has an increased susceptibility for developing multiple sclerosis will develop multiple sclerosis.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, certain changes and modifications may be practiced within the scope of the appended claims.

### Dosing Regimens

Disclosed herein are dosing regimens comprising at least one treatment cycle of an effective amount of an erythropoietin (EPO)-derived peptide followed by a rest phase. The rest phase of the dosing regimen can be a period of time where the EPO-derived peptide is not administered.

Disclosed herein are dosing regimens comprising at least one treatment cycle followed by a rest phase, wherein the treatment cycle comprises administering an effective amount of an erythropoietin (EPO)-derived peptide to allow for a sustained therapeutic effect after withdrawal of the EPO-derived peptide, wherein the EPO-derived peptide is not administered during the rest phase. Not only does an effective amount of EPO-derived peptide result in sustained therapeutic effects, but it is also an amount sufficient to cause an acute beneficial effect. Thus, the effects of the EPO-derived peptide can be measured and seen during the treatment cycle, at the end of the treatment cycle and during the rest phase. The sustained therapeutic effects are the therapeutic effects seen even after the drug effect is gone. In some aspects, the treatment cycle can comprise administering an effective amount of the EPO-derived peptide daily for 2, 3, 4, 5, 6, 7, 8, 9, 10, 11, 12, 13, or 14 days.

Disclosed herein are dosing regimens comprising at least one treatment cycle followed by a rest phase, wherein the treatment cycle comprises administering an effective amount of an erythropoietin (EPO)-derived peptide to allow for a sustained therapeutic effect after withdrawal of the EPO-derived peptide, wherein the EPO-derived peptide is not administered during the rest phase, wherein the treatment cycle comprises administering an effective amount of the EPO-derived peptide daily for 7-14 days.

Disclosed herein are dosing regimens comprising at least one treatment cycle followed by a rest phase, wherein the treatment cycle comprises administering an effective amount of an erythropoietin (EPO)-derived peptide to allow for a sustained therapeutic effect after withdrawal of the EPO-derived peptide, wherein the EPO-derived peptide is not administered during the rest phase, wherein the treatment cycle comprises administering an effective amount of the EPO-derived peptide daily for 10-12 days.

Disclosed herein are dosing regimens comprising at least one treatment cycle followed by a rest phase, wherein the treatment cycle comprises administering an effective amount of an erythropoietin (EPO)-derived peptide to allow for a sustained therapeutic effect after withdrawal of the EPO-derived peptide, wherein the EPO-derived peptide is not administered during the rest phase, wherein the treatment cycle further comprises a second treatment cycle after the rest phase.

Disclosed herein are dosing regimens comprising at least one treatment cycle followed by a rest phase, wherein the treatment cycle comprises administering an effective amount of an EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the EPO-derived peptide is not administered during the rest phase. In some aspects, the amino acid sequence GCAEHCSLNENITVPDTKV (SEQ ID NO: 1), is end protected with an acetyl group protecting the amino terminus and an amide group protecting the carboxyl terminus.

The dosing regimen can further include a second treatment cycle after the rest phase. A second rest phase can occur after the second treatment cycle. In some aspects a third, fourth, fifth, sixth, seventh, eighth, ninth or tenth treatment cycle can be administered wherein each treatment cycle is followed by a rest phase. In some aspects, the dosing regimen includes infinite treatment cycles, each followed by a rest phase. For example, a subject may be prescribed a dosing regimen that involves consecutive treatment cycles followed by rest phases for the duration of their life.

In some aspects, a second dosing regimen can be prescribed based on the re-occurrence of one or more symptoms of multiple sclerosis or other neurological deficits. The second dosing regimen can be administered 1, 2, 3, 4, 5 months or more than 5 months after the initial dosing regimen was administered. The second dosing regimen can be the same as the initial dosing regimen or can be different. For example, the initial dosing regimen can be a one week treatment cycle followed by a five month rest phase. After the five month rest phase the subject can be tested and if one or more symptoms or one or more neurological deficits have progressed or have worsened in severity or duration, then a second dosing regimen consisting of another one week treatment cycle followed by a rest phase or a two week treatment cycle followed by a rest phase can be prescribed. The dose of EPO-derived peptide can vary between the initial dosing regimen and any additionally prescribed dosing regimens. In some aspects, the one or more symptoms of multiple sclerosis can be vision loss, pain, fatigue, and impaired coordination. In some aspects, an improvement can be observed in one or more symptoms of multiple sclerosis, for example, in the severity or duration of the one or more symptoms of multiple sclerosis.

Disclosed herein are dosing regimens comprising at least one treatment cycle followed by a rest phase, wherein the treatment cycle comprises administering an effective amount of an EPO-derived peptide to allow for a sustained therapeutic effect after withdrawal of the EPO-derived peptide, wherein the EPO-derived peptide is not administered during the rest phase, wherein the treatment cycle comprises administration of an effective amount of the EPO-derived peptide daily for one week or wherein the treatment cycle comprises administration of an effective amount of the EPO-derived peptide daily for two weeks.

### Treatment Cycle

The treatment cycle can include the administration of different dosages of an EPO-derived peptide as well as administration at different time points. The EPO-derived peptide can be administered for varying amounts of time for up to one month. In some instances, the administration can occur daily for up to one, two, three, or four weeks. For example, the EPO-derived peptide can be administered daily for 1, 2, 3, 4, 5, 6, 7, 8, 9, 10, 11, 12, 13, or 14 days. In some aspects, the EPO-derived peptide can be administered daily for 1 or 2 weeks.

The length of time for each treatment cycle can vary depending on the amount of EPO-derived peptide administered per dosage. A treatment cycle can include the administration of EPO-derived peptide once, twice or three times a day. In some aspects, the EPO-derived peptide can be administered weekly. In some aspects, the Apo E mimetic can be administered once every two days or even once a week. In some instances, the EPO-derived peptide can be administered every day for 1, 2, 3, 4, 5, 6, 7, 8, 9, 10, 11, 12, 13, or 14 days. For example, the treatment cycle can include administering an EPO-derived peptide once a day for one week or daily for two weeks. Thus, each treatment cycle includes an established length of time for administration as well as an established dosing schedule during that time frame.

In some aspects, more than one EPO-derived peptide can be administered during the treatment cycles. The more than one EPO-derived peptide can be formulated together or in separate compositions. In some instances, one or more EPO-derived peptide is administered in combination with one or more other therapeutic agents, such as disease modifying drugs, including but not limited to cladribine, dimethyl fumarate, diroximel fumarate, fingolimod, monomethyl fumarate, ozanimod, siponimod, teriflunomide, interferon beta-la, interferon beta-1b, glatiramer acetate, peginterferon beta-la, alemtuzumab, mitoxantrone hydrochloride, natalizumab, ofatumumab, ponesimod, and ocerelizumab, anti-inflammatory agents, anti-spasmotic agents, immune modulating therapies, or steroids.

### Rest Phase

The disclosed dosing regimens include at least one treatment cycle followed by a rest phase. The rest phase can be a period of time wherein the EPO-derived peptide is not administered and the length of the period of time can vary. The length of the rest phase is dependent on how long the sustained therapeutic effects of the EPO-derived peptide administered during the treatment cycle last. In some aspects, the rest phase can be at least 1, 2, 3, 4, 5, 6, 7, 8, 9, 10, 11, or 12 months. In some aspects, the rest phase can be at least 1, 2, 3, 4, 5, 6, 7, 8, 9, or 10 years. For example, the rest phase can be at least one week, two weeks, three weeks or four weeks (one month).

One way to determine how long the rest phase should last is to test or evaluate the subject to determine the progression of the neurological deficits in the subject. If the neurological deficits have progressed to a level that increases the disability of subject, then the subject can be prescribed a second dosing regimen. If the neurological deficits are stable or have improved, then the rest phase can be prolonged. Subjects can be tested on a regular basis. For example, a subject can be tested every 1, 2, 3, 4, 5, 6, 7 days or every 1, 2, 3, 4, 5, 6, 7, 8, 9, 10, 11, or 12 weeks, or every 1, 2, 3, 4, 5, 6, 7, 8, 9, 10, 11, 12, 14, 16, 18, 24, 30 or 36 months.

In some aspects, the rest phase can be decreased or extended depending on the dose of EPO-derived peptide administered and the reduction in neurological deficits achieved during the treatment cycle. For example, the rest phase can be extended if the dose of EPO-derived peptide during the treatment cycle is increased and the neurological deficits are substantially reduced and/or the clinical scoring system is substantially improved. The length of the rest phase can also vary based on the length of the treatment cycle. For instance, if a subject receives a certain dose of EPO-derived peptide daily for one week then the rest phase may be shorter than a subject that receives the same dose of EPO-derived peptide once a week for two weeks. Table 2 provides the clinical scoring system for quantifying neurological deficits.

In some aspects, the subject can be assessed using a neurological exam, am expanded disability status scale (EDSS), via imaging and a timed 25 foot walk. For example, neurologic deficits can be determined using a neurlogic exam in which the findings deviate from normal.

In some aspects, although an EPO-derived peptide is not administered during the rest phase, a multiple sclerosis therapeutic other than an EPO-derived peptide can be administered during the rest phase.

In some aspects, the beneficial effects of the EPO-derived peptide can still be present in a subject even after the treatment cycle is complete. In some aspects, the EPO-derived peptide is no longer detectable in a subject after the treatment cycle is complete. Thus, the long-term therapeutic effects are not from residual EPO-derived peptide.

### Dose

The dose or dosage of EPO-derived peptides can vary depending on many factors, such as but not limited to, the route of administration, the formulation, the severity of the patient's condition/disease, previous treatments, the patient's size, weight, surface area, age, and gender, other drugs being administered, and the overall general health of the patient including the presence or absence of other diseases, disorders or illnesses, length of treatment cycle, or whether other drugs are included in the regimen, and can be determined by one of skill in the art. The particular dosage of a pharmaceutical composition to be administered to the patient will depend on a variety of considerations (e.g., the severity of the symptoms of the disease, disorder or condition), the age and physical characteristics of the subject and other considerations known to those of ordinary skill in the art. Variations in the needed dosage may be expected. Variations in dosage levels can be adjusted using standard empirical routes for optimization. Dosages can be established using clinical approaches known to one of ordinary skill in the art. Administrations of the compositions described herein can be single or multiple (e.g., 2- or 3-, 4-, 6-, 8-, 10-, 20-, 50-, 100-, 150-, or more fold).

Effective dosages can be determined empirically, and making such determinations is within the skill in the art. The dosage ranges for the administration of the compositions are those large enough to produce the desired effect in which the disease is treated. For example, the dosage can be an amount effective to provide therapeutic effects and provide or allow for sustained therapeutic effects even after the treatment (i.e., EPO-derived peptide) is withdrawn. The therapeutic effects can be, but are not limited to, reducing A1 astrocyte activation in spinal cord, decreasing complement component C3, reducing and/or returning elevated mononuclear cell counts to normal, decreasing the number of dendritic cells, decreasing proinflammatory cytokines (e.g., IL-2, IL-6, TNF-alpha and INF-gamma), expand Treg cells, and reduce the number of T helper Th17 positive cells. The therapeutic effects can be measured by markers of neuroinflammation. The therapeutic effects can be measured by radiographic imaging (e.g., CT scan, MRI (with or without contrast)), Expanded Disability Status Scale, bioluminescence imaging, serum biomarkers, for example, non phosphorylated neurofilament protein, neurologic examination or other known methods.

The dosage should not be so large as to cause adverse side effects, such as unwanted cross-reactions, anaphylactic reactions, and the like. The dosage can be adjusted by the individual physician in the event of any counter-indications. Dosage can vary, and can be administered in one or more dose administrations daily, for one or several days. Guidance can be found in the literature for appropriate dosages for given classes of pharmaceutical products.

Suitable dosages include, but are not limited to amounts between 0.01 mg/kg and 10 mg/kg. For example, disclosed herein are methods involving administering one or more of the disclosed EPO-derived peptide to a subject, wherein the EPO-derived peptide is administered in an amount of about 0.15 mg/kg to about 5 mg/kg. In some aspects, the EPO-derived peptide can be administered in an amount of about 5, 6, 7, 8, 9, or 10 mg or any amount in between daily.

The EPO-derived peptide dose can be administered as a bolus injection or as an infusion over one or more hours.

### EPO-Derived Peptides

Erythropoietin (EPO) is a pleiotropic cytokine involved in the proliferation, viability, and terminal differentiation of erythroid precursor cells (Bunn F. Erythropoietin. Cold Spring Harb Perspect Med 2013:3:a011619; and Martinez F and Pallet N. Journal of the American Society of Nephrology 2014:9: 1887-1889). Whole-molecule EPO provides neuroprotection against ischemic toxicity, ameliorates brain injury, and improves memory in animal models by preventing beta-amyloid degradation (Lee S T, et al. Journal of Neurochemistry 2012:120:115-124; Shang Y C, et al. Aging 2012:4:187-201: Li Q, et al. Life Sciences 2017:194:15-25; and Wei S, Luo C, Yu S, et al. Experimental Cell Research 2017:361:342-352). Given the potential healing role of EPO in the development of new therapeutic approaches for neuroinflammatory disorders, research efforts remain underway to more clearly define its mechanism and its downstream effects (Maiese K, et al. Erythropoietin: New Directions for the Nervous System 2012: 13:11102-11129).

Examples of EPO-derived peptides are provided in Table 1.

TABLE 1
EPO-derived peptides.

| Name | Sequence | SEQ ID NO |
|---|---|---|
| JM-4 | GCAEHCSLNENITVPDTKV | 1 |
| JM-5 | CAEHCSLNENITVP | 2 |
| JM-7 | TTGCAEHCSLNENITVP | 3 |
| JM-1L | CAEHCSLNENITVPDTKV | 4 |
| JM-6 | TTGCAEHCSLNENITVPDTKV | 5 |
| JM-7 | TTGCAEHCSLNENITVP | 6 |
| BW2L | CAEHCSLKHQGLNKNINLDSVDGVP | 7 |
| BW3L | GCAEHCSLMENNLRRPNL | 8 |
| BW4 | AEHCSLMENNLRRPNL | 9 |

The term “erythropoietin” (EPO) refers to the principal hormone involved in the regulation of erythrocyte differentiation and the maintenance of a physiological level of circulating erythrocyte mass. The EPO molecule is an 193 amino acid peptide having amino acid sequence (SEQ ID NO: 10)
MGVHECPAWLWLLLSLLSLPLGLPVLGAPPRLICDSRVLERYLLEAKEAE NITTGCAEHCSLNENI TVPDTKVNFYAWKRMEVGQQAVEVWQGLALLSEA VLRGQALLVNSSQPWEPLQLHVDKAVSGLRSLTTLLRALGAQKEAISPPD AASAAPLRTI TADTFRKLFRVYSNFLRGKLKLYTGEACRTGDR
   that is further processed into a mature form.

The EPO molecule comprises: 1) signal peptide (positions 1-27) having amino acid sequence (SEQ ID NO: 11) MGVHECPAWLWLLLSLLSLPLGLPVLG: 2) chain (positions 28-193) having amino acid sequence (SEQ ID NO: 12) APPRLICDSRVLERYLLEAKEAENI TTGCAEHCSLNENITVPDTKVNFYA WKRMEVGQQAVEVWQGLALLS EAVLRGQALLVNS SQPWEPLQLHVDKAVS GLRSLTTLLRALGAQKEAI SPPDAASAAPLRTITADTFRKLFRVYSNFLR GKLKLYTGEACRTGDR: 3) propeptide (positions 190-193) having amino acid sequence TGDR (SEQ ID NO: 13); and 4) propeptide (position 193) (R).

The terms “whole EPO” and “whole EPO molecule” are used interchangeably herein to refer to the 165 amino acid peptide backbone (chain) of recombinant EPO protein, having substantial identity to amino acid sequence (SEQ ID NO: 14) APPRLICDSRVLERYLLEAKEAENI TTGCAEHCSLNENITVPNTKVNFYA WKRMEVGQQAVEVWQGLALLS EAVLRGQALLVNS SQPWEPLQLHVDLAVS GLRELTTLLRALGAQLEAISPPDAASAAPLATITANTERKLERVYSNALR GKLKLYTQEACRTGD. This backbone contains three N-linked carbohydrates attached to Asp24, Asp38, and Asp83 and one O-linked carbohydrate attached to Ser126. (see, Browne, J K, et al., Erythropoietin: gene cloning, protein structure, and biological properties. Cold Spring Harb. Symp. Quant. Biol. 51:693-702, 1986; the contents of which are incorporated herein by reference in their entirety).

### Methods of Treatment

The compositions, dosing regimes and methods disclosed herein can be useful for the treatment of a subject with multiple sclerosis. For example, disclosed herein are methods of treating multiple sclerosis, relapsing-remitting multiple sclerosis, reducing A1 astrocyte activation in spinal cord, decreasing complement component C3, and treating a disease, disorder or condition having an inflammatory or autoimmune component by administering an effective amount of an EPO-derived peptide for at least one treatment cycle followed by a rest phase. Thus, the disclosed methods can involve administering an EPO-derived peptide using one or more of the disclosed dosing regimens. Thus, any of the disclosed treatment cycles or rest phases can be used in the disclosed methods. The methods disclosed herein can allow for prolonged therapeutic effects even in the absence of the therapeutic. The disclosed methods can include the administration of an effective amount of EPO-derived peptide. The effective amount of an EPO-derived peptide can be an amount that allows for sustained therapeutic effects after the EPO-derived peptide has been withdrawn.

In some aspects, the disease, disorder or condition having an inflammatory or autoimmune component can be dementia, acute cerebrovascular injury, acute spinal cord injury, acute traumatic brain injury and repetitive mild traumatic brain injury, acute cardiovascular injury, arthritis, autoimmune disease, demyelinating disease, a stroke, multiple sclerosis, a neurological injury and immune-mediated inflammation.

Disclosed herein are methods of treating multiple sclerosis, the methods comprising administering to a subject an effective amount of an erythropoietin (EPO)-derived peptide for at least one treatment cycle, wherein the treatment cycle comprises an effective amount of the EPO-derived peptide to allow for a sustained therapeutic effect after withdrawal of the EPO-derived peptide, wherein the treatment cycle is followed by a rest phase, and wherein the EPO-derived peptide is not administered during the rest phase. In some aspects, the EPO-derived peptide consists of the amino acid sequence GCAEHCSLNENITVPDTKV (SEQ ID NO: 1).

Disclosed herein are methods of treating relapsing-remitting multiple sclerosis, the methods comprising administering to a subject an effective amount of an erythropoietin (EPO)-derived peptide for at least one treatment cycle, wherein the treatment cycle comprises an effective amount of the EPO-derived peptide to allow for a sustained therapeutic effect after withdrawal of the EPO-derived peptide, wherein the treatment cycle is followed by a rest phase, and wherein the EPO-derived peptide is not administered during the rest phase. In some aspects, the EPO-derived peptide consists of the amino acid sequence GCAEHCSLNENITVPDTKV (SEQ ID NO: 1).

Disclosed herein are methods of treating relapsing-remitting multiple sclerosis, the methods comprising administering to a subject an effective amount of an erythropoietin (EPO)-derived peptide for at least one treatment cycle, wherein the treatment cycle comprises administering an effective amount of the EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the treatment cycle is followed by a rest phase, wherein the EPO-derived peptide is not administered during the rest phase.

Disclosed herein are methods of reducing A1 astrocyte activation in spinal cord, the methods comprising administering to a subject an effective amount of an erythropoietin (EPO)-derived peptide for at least one treatment cycle, wherein the treatment cycle comprises administering an effective amount of the EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the treatment cycle is followed by a rest phase, wherein the EPO-derived peptide is not administered during the rest phase.

Disclosed herein are methods of decreasing complement component C3, the methods comprising administering to a subject an effective amount of an erythropoietin (EPO)-derived peptide for at least one treatment cycle, wherein the treatment cycle comprises administering an effective amount of the EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the treatment cycle is followed by a rest phase, wherein the EPO-derived peptide is not administered during the rest phase.

Disclosed herein are methods of treating a disease, disorder or condition having an inflammatory or autoimmune component in a subject in need thereof, the methods comprising administering to a subject an effective amount of an erythropoietin (EPO)-derived peptide for at least one treatment cycle, wherein the treatment cycle comprises administering an effective amount of the EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the treatment cycle is followed by a rest phase, wherein the EPO-derived peptide is not administered during the rest phase, wherein the composition is effective at ameliorating at least one symptom from at least one disease, disorder, or condition having an inflammatory or autoimmune component. In some aspects, the disease, disorder or condition having an inflammatory or autoimmune component can be dementia, acute cerebrovascular injury, acute spinal cord injury, acute traumatic brain injury and repetitive mild traumatic brain injury, acute cardiovascular injury, arthritis, autoimmune disease, demyelinating disease, a stroke, multiple sclerosis, a neurological injury and immune-mediated inflammation.

Any of the disclosed EPO-derived peptides can be used in the disclosed methods. For example, the EPO-derived peptide can be GCAEHCSLNENITVPDTKV (SEQ ID NO: 1; JM-4), in particular the EPO-derived peptide can be the JM-4 peptide that has an acetyl group on the amino terminus and an amide group at the carboxyl terminus.

Also disclosed herein are method of using the EPO-derived peptides. For example, disclosed herein are uses of an erythropoietin (EPO)-derived peptide for the production of a medicament for the treatment of multiple sclerosis (MS) in a patient. In some aspects, the treatment can comprise a first treatment cycle of the EPO-derived peptide followed by at least one further treatment cycle of the EPO-derived peptide, in which each treatment cycle comprises 1-14 doses which are applied on consecutive days, wherein the daily dose is >0) and ≤10 mg, and wherein each treatment cycle is separated from the next treatment cycle by at least 1-24 months. In some aspects, the at least one further treatment cycle can be administered at least 5 months after the first treatment cycle. In some aspects, the at least one further treatment cycle can be at the same daily dose for a shorter duration than the first treatment cycle. In some aspects, the first treatment cycle of the EPO-derived peptide can be at a dose of 5, 6, 7, 8, 9, or 10 mg/day for five days. In some aspects, the patient can be retreated at 12 months after the first treatment cycle with a further treatment cycle of the EPO-derived peptide at a dose of 6, 7, 8, 9, or 10 mg/day for five days. In some aspects, the two initial treatment cycles of the EPO-derived peptide can be e followed by a third or subsequent treatment cycle of the EPO-derived peptide only upon evidence of renewed MS activity. In some aspects, he third or subsequent treatment cycle can be at a dose of 5-10 mg/day for 1 week. In some aspects, the evidence of renewed MS activity can be diagnosed by clinical means. In some aspects, the clinical means can be selected from the group consisting of relapse or progression of neurological disability. In some aspects, the evidence of renewed MS activity can be diagnosed by magnetic resonance imaging (MRI) of the brain or spinal cord. In some aspects, the MS activity detected by MRI is indicated by the occurrence of new cerebral or spinal lesions on T1 or T2 weighted images or by an increase in lesional gadolinium uptake or the increase in volume of such lesions. In some aspects, the repeated MRIs are performed at fixed intervals after the second treatment cycle of the EPO-derived peptide in order to determine whether a third or subsequent treatment cycle of the EPO-derived peptide is necessary. In some aspects, the third or subsequent treatment cycle of the EPO-derived peptide can be performed before the disease re-manifests clinically. In some aspects, the EPO-derived peptide can be administered intravenously. In some aspects, the MS can be relapsing MS. In some aspects, the patient has received prior therapy for MS. In some aspects, the EPO-derived peptide consists of the amino acid sequence GCAEHCSLNENITVPDTKV (SEQ ID NO: 1).

The disclosed methods of treating can occur at different times depending on the subject. In particular, treatment can occur in a subject considered to be of high or high residual risk of disability due to MS or relapsing-remitting multiple sclerosis. In some aspects, the treatment can be initiated after a subject is stabilized following an acute MS attack. An acute MS attack can include an exacerbation of MS resulting in one or more new symptoms or the worsening of old symptoms. In some aspects, the treatment can be initiated immediately after the acute MS attack, or 2, 4, 6, 8, 10, 12 weeks or 2, 4, 6, 8, 10, or 12 months after the acute MS attack. The treatment can be initiated following a blood test, lumbar puncture or an MRI. In some aspects, the MRI may reveal one or more (new) brain or spinal cord lesions. Subjects considered as high risk for MS can be those individuals that have abnormal responses by the body's immune system that may cause inflammation and damage in the central nervous system, exposure to environmental toxins, low vitamin D levels, smoking, obesity, previous infection with Epstein-Barr virus, genetic factors, and being female. In high risk subjects, treatment can be extended.

In some aspects, the methods can further include the step of identifying a subject (e.g., a human patient) who has multiple sclerosis, relapsing-remitting multiple sclerosis, or a disease, disorder or condition having an inflammatory or autoimmune component and then providing to the subject a composition comprising the one or more of the EPO-derived peptides as disclosed herein. In some aspects, the subject has multiple sclerosis. In some aspects, the subject has a disease, disorder or condition having an inflammatory or autoimmune component, wherein the disease, disorder or condition having an inflammatory or autoimmune component is dementia, acute cerebrovascular injury, acute spinal cord injury, acute traumatic brain injury and repetitive mild traumatic brain injury, acute cardiovascular injury, arthritis, autoimmune disease, demyelinating disease, a stroke, multiple sclerosis, a neurological injury and immune-mediated inflammation.

Treatment cycles. The treatment cycles, as described herein with respect to the dosing regimens, can vary in length of time. In some aspects, the treatment cycle can be at least one or two weeks but can last up to one month. In some aspects, the disclosed methods have a treatment cycle that involves the administration of an effective amount of an EPO-derived peptide daily for one, two, three, or four weeks. A treatment cycle can include the administration of EPO-derived peptide daily for one week or daily for two weeks. In some aspects, the EPO-derived peptide can be administered daily or multiple times in a single day. In some aspects, the EPO-derived peptide can be administered once every two weeks or even once a month. In some instances, the EPO-derived peptide can be administered every day for 1, 2, 3, 4, 5, 6, 7, 8, 9, 10, 11, 12, 13, or 14 days. Each treatment cycle can include an established length of time for administration as well as an established dosing schedule during that time frame.

The methods can further include a second treatment cycle after the rest phase. In some aspects, the second treatment cycle can be administered after a four week rest phase. In some aspects, the second treatment cycle can be administered at least one year from the beginning of the initial treatment cycle.

Rest phase. In some aspects, the rest phase, as previously described herein with regards to the dosing regimen, can be at least one week but can last for several years. An EPO-derived peptide is not administered during the rest phase.

The length of the rest phase is dependent on how long the sustained therapeutic effects of the EPO-derived peptide administered during the treatment cycle last. In some aspects, the rest phase can be at least 1, 2, 3, 4, 5, 6, 7, 8, 9, 10, 11, or 12 months. In some aspects, the rest phase can be at least 1, 2, 3, 4, 5, 6, 7, 8, 9, or 10 years. For example, the rest phase can be at least one week, two weeks, three weeks or four weeks (one month).

In some aspects, the rest phase can be decreased or extended depending on the dose of EPO-derived peptide administered during the treatment cycle. For example, the rest phase can be extended if the dose of EPO-derived peptide during the treatment cycle is increased. The length of the rest phase can also vary based on the length of the treatment cycle. For instance, if a subject receives a certain dose of the EPO-derived peptide daily for one week then the rest phase may be shorter than a subject that receives the same dose of the EPO-derived peptide daily for two weeks for six months.

Although an EPO-derived peptide is not administered during the rest phase, a multiple sclerosis therapeutic other than an EPO-derived peptide can be administered during the rest phase. The multiple sclerosis therapeutic other than an EPO-derived peptide can be any disease modifying therapy approved for use in multiple sclerosis.

### EAE Animal Model and Multiple Sclerosis

Experimental autoimmune encephalomyelitis (EAE) is a central nervous system inflammatory demyelinating disease involving acute injury to the brain and spinal cord white matter. This animal model has been used widely by many investigators to study disease pathogenesis and to explore new therapies for its human counterpart, multiple sclerosis (MS). Pathogenesis of both MS and EAE is believed to involve (1) activation of myelin reactive T cells: (2) upregulated expression of chemokines and adhesion molecules; (3) focal T cells and macrophage infiltration into the CNS white matter; and (4) demyelination and axonal injury and loss of neurological function (Trapp., B. et al., J Neuroimmunol, 98: 49-56 (1999)). In both EAE and MS, activated T-lymphocytes specific for self-antigens present in myelin are linked to CNS inflammation and to the breakdown of the blood brain barrier to peripheral blood leukocytes and plasma proteins: this is predominantly restricted to myelin rich white matter area of the CNS (Bettelli, E., et al., J Exp Med, 197: 1073-81 (2003): Crawford, M. P., et al., Blood 103(11): 4222-31 (2004); Abdul-Maj id, K. B., et al., J Neuroimmunol, 141: 10-19 (2003); Battistini, L., et al., Blood, 101: 4775-82 (2003)).

EAE can be induced experimentally in genetically susceptible animals, such as mice, by immunization with immunodominant peptides from myelin proteins, such as myelin basic protein (MBP), proteolipid protein (PLP), and myelin oligodendrocytes glycoprotein (MOG), emulsified in complete Freund's adjuvant followed by injection of pertussis toxin as an additional adjuvant for certain mouse strains (Li, W., et al., Ann Neurol, 56: 767-77 (2004)). Disease development is variable from strain to strain. For example, in SJL/J mice, PLP or MBP induces a relapsing remitting progression, whereas C57BL/6 mice immunized with MOG often develop a chronic form of disease.

Multiple sclerosis can include relapsing-remitting MS, secondary progressive MS, primary progressive MS and primary relapsing MS.

### Delivery

Disclosed herein are EPO-derived peptides, and compositions comprising EPO-derived peptides. The therapeutically effective amount can be the amount of the composition administered to a subject that leads to a full resolution of the symptoms of the condition or disease, a reduction in the severity of the symptoms of the condition or disease, or a slowing of the progression of symptoms of the condition or disease. The methods described herein can also include a monitoring step to optimize dosing. The compositions described herein can be administered as a preventive treatment or to delay or slow the progression of degenerative changes.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with multiple sclerosis, dementia, acute cerebrovascular injury, acute spinal cord injury, acute traumatic brain injury and repetitive mild traumatic brain injury, acute cardiovascular injury, arthritis, autoimmune disease, demyelinating disease, a stroke, multiple sclerosis, or a neurological injury and immune-mediated inflammation in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the disease, disorder or condition is delayed, hindered, or prevented, or the disease, disorder or condition or a symptom of the disease, disorder or condition is ameliorated. One or more of the symptoms can be less severe. Recovery can be accelerated in an individual who has been treated.

In the methods described herein, administration or delivery of the EPO-derived peptide can be via a variety of mechanisms. As defined above, disclosed herein are dosing regimens and methods of using those dosing regimens to treat multiple sclerosis. The dosing regimens and methods include compositions containing any one or more of the EPO-derived peptide described herein that can also include a carrier such as a pharmaceutically acceptable carrier. Also disclosed herein are pharmaceutical compositions, comprising the EPO-derived peptide disclosed herein, and a pharmaceutically acceptable carrier.

In some aspects, the compositions disclosed herein can be used for direct delivery of modified therapeutic cells.

The disclosed EPO-derived peptides can be in solution or in suspension (for example, incorporated into microparticles, liposomes, or cells).

Any suitable route of administration can be used for the disclosed compositions. Suitable routes of administration can, for example, include topical, enteral, local, systemic, or parenteral. For example, administration can be epicutaneous, inhalational, enema, conjunctival, eye drops, ear drops, alveolar, nasal, intranasal, enteral, oral, intraoral, transoral, intestinal, rectal, intrarectal, transrectal, injection, infusion, intravenous, intraarterial, intramuscular, intracerebral, intraventricular, intracerebroventricular, intracardiac, subcutaneous, intraosseous, intradermal, intrathecal, intraperitoneal, intravesical, intracavernosal, intramedullar, intraocular, intracranial, transdermal, transmucosal, transnasal, inhalational, intracisternal, epidural, peridural, intravitreal, etc. The disclosed compositions can be used in and with any other therapy.

The compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, P A 1995. Typically, an appropriate amount of pharmaceutically acceptable salt is used in the formulation to render the formulation isotonic. Other examples of the pharmaceutically acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions may also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

Unlike typical peptide formulations, the peptides of this invention comprising D-form amino acids can be administered, even orally, without protection against proteolysis by stomach acid, etc. Nevertheless, in some aspects, peptide delivery can be enhanced by the use of protective excipients. This is typically accomplished either by complexing the polypeptide with a composition to render it resistant to acidic and enzymatic hydrolysis or by packaging the polypeptide in an appropriately resistant carrier such as a liposome. Means of protecting polypeptides for oral delivery are well known in the art (see, e.g., U.S. Pat. No. 5,391,377 describing lipid compositions for oral delivery of therapeutic agents).

Elevated serum half-life can be maintained by the use of sustained-release protein “packaging” systems. Such sustained release systems are well known to those of skill in the art. In some aspects, the ProLease biodegradable microsphere delivery system for proteins and peptides (Tracy (1998) Biotechnol. Prog., 14: 108: Johnson et al. (1996) Nature Med. 2: 795: Herbert et al. (1998), Pharmaceut. Res. 15, 357) a dry powder composed of biodegradable polymeric microspheres containing the active agent in a polymer matrix that can be compounded as a dry formulation with or without other agents.

The ProLease microsphere fabrication process was specifically designed to achieve a high encapsulation efficiency while maintaining integrity of the active agent. The process consists of (i) preparation of freeze-dried drug particles from bulk by spray freeze-drying the drug solution with stabilizing excipients, (ii) preparation of a drug-polymer suspension followed by sonication or homogenization to reduce the drug particle size, (iii) production of frozen drug-polymer microspheres by atomization into liquid nitrogen, (iv) extraction of the polymer solvent with ethanol, and (v) filtration and vacuum drying to produce the final dry-powder product. The resulting powder contains the solid form of the active agents, which is homogeneously and rigidly dispersed within porous polymer particles. The polymer most commonly used in the process, poly(lactide-co-glycolide) (PLG), is both biocompatible and biodegradable.

Encapsulation can be achieved at low temperatures (e.g., −40° C.). During encapsulation, the protein is maintained in the solid state in the absence of water, thus minimizing water-induced conformational mobility of the protein, preventing protein degradation reactions that include water as a reactant, and avoiding organic-aqueous interfaces where proteins may undergo denaturation. A preferred process uses solvents in which most proteins are insoluble, thus yielding high encapsulation efficiencies (e.g., greater than 95%).

In some aspects, one or more components of the solution can be provided as a “concentrate”, e.g., in a storage container (e.g., in a premeasured volume) ready for dilution, or in a soluble capsule ready for addition to a volume of water.

The foregoing formulations and administration methods are intended to be illustrative and not limiting. It will be appreciated that, using the teaching provided herein, other suitable formulations and modes of administration can be readily devised.

### Combination Therapy

In some aspects of the disclosed methods, the EPO-derived peptides can be administered alone or in combination with one or more additional therapeutic agents. The additional therapeutic agents are selected based on the disease or symptom to be treated. A description of the various classes of suitable pharmacological agents and drugs may be found in Goodman and Gilman, The Pharmacological Basis of Therapeutics, (11th Ed., McGraw-Hill Publishing Co.) (2005). For example, pharmaceutical compositions containing EPO-derived peptides can be administered in combination with one or more known therapeutic agents for treating multiple sclerosis. Therapeutic agents for treating multiple sclerosis include, but are not limited to, anti-inflammatory agents, anti-spasmotic agents, immune modulating therapies, steroids, and disease modifying drugs. Examples of disease modifying drugs include, but are not limited to, cladribine, dimethyl fumarate, diroximel fumarate, fingolimod, monomethyl fumarate, ozanimod, siponimod, teriflunomide, interferon beta-la, interferon beta-1b, glatiramer acetate, peginterferon beta-la, alemtuzumab, mitoxantrone hydrochloride, natalizumab, ofatumumab, ponesimod, and ocerelizumab.

The combination therapies can include administering the EPO-derived peptide and an additional therapeutic agent during the treatment cycle of a dosing regimen. The combination therapies can also include administering the EPO-derived peptides during the treatment cycle and an additional therapeutic agent during the rest phase.

### Pharmaceutical Compositions

Disclosed herein are pharmaceutical compositions comprising the compositions disclosed herein. In some aspects, the pharmaceutical composition can comprise any of EPO-derived peptides disclosed herein. For example, disclosed herein are pharmaceutical compositions comprising one or more of the EPO-derived peptides listed in Table 1. In some aspects, the pharmaceutical compositions further comprise a pharmaceutically acceptable carrier.

As used herein, the term “pharmaceutically acceptable carrier” refers to solvents, dispersion media, coatings, antibacterial, isotonic and absorption delaying agents, buffers, excipients, binders, lubricants, gels, surfactants that can be used as media for a pharmaceutically acceptable substance. The pharmaceutically acceptable carriers can be lipid-based or a polymer-based colloid. Examples of colloids include liposomes, hydrogels, microparticles, nanoparticles and micelles. The compositions can be formulated for administration by any of a variety of routes of administration, and can include one or more physiologically acceptable excipients, which can vary depending on the route of administration. Any of the EPO-derived peptides or other drugs described herein can be administered in the form of a pharmaceutical composition.

As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed. The compositions can also include additional agents (e.g., preservatives).

The pharmaceutical compositions as disclosed herein can be prepared for oral or parenteral administration. Pharmaceutical compositions prepared for parenteral administration include those prepared for intravenous (or intra-arterial), intramuscular, subcutaneous, intrathecal, transmucosal (e.g., intranasal) direct or local injection, transdermal (e.g., topical) or intraperitoneal administration. Aerosol inhalation can also be used. Paternal administration can be in the form of a single bolus dose, or may be, for example, by a continuous pump. In some aspects, the local or direct injection can be via convection enhanced delivery. In some aspects, the compositions can be prepared for parenteral administration that includes dissolving or suspending any of the MSUT2 inhibitors disclosed herein in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

In some aspects, the compositions disclosed herein are formulated for oral, intramuscular, intravenous, subcutaneous, intrathecal, direct or local injection, intranasal, or intraperitoneal administration.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment. The compositions can also be formulated as powders, elixirs, suspensions, emulsions, solutions, syrups, aerosols, lotions, creams, ointments, gels, suppositories, sterile injectable solutions and sterile packaged powders. As used herein “pharmaceutically acceptable” means molecules and compositions that do not produce or lead to an untoward reaction (i.e., adverse, negative or allergic reaction) when administered to a subject as intended (i.e., as appropriate).

The compositions as disclosed herein can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery. In some aspects, the route of administration includes but is not limited to direct injection into the brain. Such administration can be done without surgery, or with surgery.

### Kits

The kits described herein can include any combination of the compositions (e.g., one or more of the EPO-derived peptides) described above and suitable instructions (e.g., written and/or provided as audio-, visual-, or audiovisual material). In some aspects, the kit comprises a predetermined amount of a composition comprising any one compositions disclosed herein. The kit can further comprise one or more of the following: instructions, sterile fluid, syringes, a sterile container, delivery devices, and buffers or other control reagents.

### EXAMPLES

### Example 1: Prolonged Beneficial Effect of Brief Erythropoietin Peptide JM4 Therapy on Chronic Relapsing EAE

Experimental autoimmune encephalomyelitis (EAE) is a T cell mediated autoimmune disease affecting the central nervous system (CNS) in laboratory animals that leads to progressive paralysis closely resembling clinical manifestations of Multiple Sclerosis (MS) (Robinson A, et al. Handbook of Clinical Neurology 2014: 122:173-89). Due to the clinical and immunopathological similarities to MS, EAE is an animal model widely used in the development of therapies for MS (Robinson A, et al. Handbook of Clinical Neurology 2014: 122:173-89). Two newly recognized FDA-approved MS therapies known as pulsed immune reconstitution therapies, alemtuzumab and cladribine, follow a brief treatment course to induce sustained clinical and radiographic beneficial effects that may last for many years (Sorensen P and Sellebjerg F. Therapeutic Advances in Neurological Disorders 2019:12:1-16).

Results of preclinical studies in a short term myelin oligodendrocyte glycoprotein (MOG) EAE mouse model revealed potent immunomodulatory effects of EPO by demonstrating marked improvement in weakness, reduced mononuclear cell infiltration and downregulation of glial Major Histocompatibility Complex (MHC) class II expression within the inflamed CNS (Yuan R, et al. PLOS ONE 2008:4:3). Although whole-molecule EPO shows therapeutic promise in the EAE model, toxicities including elevation in red cell mass, cardiovascular complications, stroke and hypertension, limit interest in its use in human clinical practice (Dicato M. Oncologist 2008:3:11-15; Ehrenreich H, et al. Stroke 2009:40:e647-e656; and Corwin H, et al. A. New England Journal of Medicine 2007:357:965-76). To avoid the side effects induced by whole-molecule EPO, a small side-effect free EPO-derived peptide, JM4, was generated and found to have robust EPO-like tissue protective properties in symptomatic EAE mice, without the activities responsible for excessive erythropoiesis (Yuan R, et al. Neurotherapeutics 2015: 12:850-861; and Wang B, et al. Neurotherapeutics 2016:2:418-427).

It was found that treatment of EAE mice with both whole molecule EPO and JM4 reduced elevated mononuclear cells count to normal, decreased dendritic cells by ten-fold, and decreased proinflammatory cytokines including IL-2, IL-6, TNF-alpha and INF-gamma (Yuan R, et al. Neurotherapeutics 2015: 12:850-861). Additionally, JM4 peptide was found to expand Treg cells and reduce T helper Th 17 positive cells in SJL/J EAE mice (Yuan R, et al. Neurotherapeutics 2015: 12:850-861). These findings, suggest a mechanism of JM4 activity in neuroprotection that closely resembles that of whole molecule EPO through profound effects on both innate and acquired immunity, involving microglia, astrocytes and T-cells.

Bioluminescence imaging (BLI) is a sensitive quantitative imaging modality that may be utilized in EAE to non-invasively monitor neuroinflammation, predict disease onset and follow disease flareups (Luo J, et al. Journal of Neuroinflammation 2008:5:6). BLI has shown widespread clinical applicability to track the progression of amyloid beta accumulation in Alzheimer's mouse models and to measure prion infectivity (Watts J, et al. PNAS 2011:108:2528-2533; and Tamguney G, et al. PNAS 2009:106:15002-15006). Prior work has used BLI to serially quantify glial fibrillary acidic protein (GFAP) expression, a known marker for astrocyte activity, to follow CNS neuroinflammation in transgenic mouse models, thereby serving as a paradigm for monitoring the inflammatory disease process (Luo J, et al. Journal of Neuroinflammation 2008:5:6; Hochgraefe K and Mandelkow E M. Molecular Neurobiology 2013:47:868-882; and Zhu L, et al. Neuroscience Letters 2004:367:210-212). Astrocyte activation is a practical biomarker that reflects the process for EAE induction and is observed prior to the onset of clinical symptoms (Brambilla R. Acta Neuropathology 2019:137:757-783). Elevated GFAP expression is seen before the onset of clinical symptoms in relapsing remitting EAE mice, and astrocyte reactivity similarly occurs in the beginning stages of MS lesion formation and persists chronically (Brambilla R. Acta Neuropathology 2019:137:757-783; and Alvarez J, et al. Glia 2013:61:1939-58). In recent years, two different types of reactive astrocytes termed A1 and A2 have been identified (Liddelow S, et al. Nature 2017:541:481-487; and Smith M, et al. Brain Research 1983:264:241-53). A1 astrocytes overexpress GFAP, present with upregulated expression of complement component C3, and are upregulated in MS and neurodegenerative disorders (Li W, et al. Annals of Neurology 2004:56:767-77; and Smith M, et al. Brain Research 1983:264:241-53), where they lead to death of oligodendrocytes and neurons. A2 astrocytes in contrast are thought to be neuroprotective (Liddelow S, et al. Nature 2017:541:481-487).

The results described herein show that JM4 treatment substantially reduces inflammatory effects long term in the acute MOG monophasic disease and in chronic relapsing-remitting proteolipid protein (PLP) induced EAE mice, and that BLI is a good methodology for tracking the clinical course of EAE. The results also demonstrate increased C3 expression in the CNS of EAE mice and show marked reduction in C3 expression in JM4 treated animals. These sustained and long-term clinical benefits of brief treatment with JM4 resemble those of the newly recognized immune reconstitution multiple sclerosis drugs, alemtuzumab and cladribine.

Methods. Animals. Male FVB/N-Tg (GFAP-luc+/−) mice (Xenogen Corp, Alameda, CA) were crossed with either albino C57BL/6J-Tyrc-2j or SJL/J mice purchased from Charles River Laboratories (Wilmington, MA). The FI offspring were genotyped by PCR following the manufacturer's protocol. Female mice between 8-10 weeks of age positive for GFAP (GFAP-luc/C57 or GFAP-Luc/SJL) expression were used.

Peptides. The myelin-derived antigen proteolipid protein peptide PLP139-151 (HSLGKWLGHPDKF: SEQ ID NO: 15) or MOG35-55 (MEVGWYRSPFSRVVHLYRNGK: SEQ ID NO: 11) was used for the induction of relapsing-remitting chronic and the monophasic forms of EAE, respectively. A non-hematopoietic (EPO)-derived short peptide fragment, JM4 (GCAEHCSLNENITVPDTKV; SEQ ID NO: 1), was used for therapy. Peptides were purchased from United Biochemical Research, Inc., WA.

Induction of EAE and clinical assessment of animals. Active EAE was induced according to a standard protocol (Smith M, et al. Brain Research 1983:264:241-53). Mice were immunized on Day 0 by subcutaneous injection on both sides of the tail base with 100 μL of an emulsion composed of either PLP139-151 or MOG35-55 peptide in an equal volume of Freund's adjuvant, supplemented with 4 mg/mL killed M. tuberculosis H37Ra (Difco Laboratories, Detroit, MI). The initial dose of antigen was 100 μg of PLP peptide for inducing relapsing EAE in SJL/J mice, or 200 μg of MOG peptide per C57BL/6 mouse for induction of acute monophasic EAE. GFAP-Luc/SJL/J mice received a second PLP antigen immunization on Day 7. Immediately following these immunizations, mice received intravenous (IV) injections of 200 ng Bordetella pertussis toxin diluted in 200 μL phosphate buffered saline (PBS) (List Biological Laboratories, Campbell, CA). The GFAP-Luc/C57BL mice received an additional IV injection of 200 ng Bordetella pertussis toxin on post-inoculation Day 2. Animals were weighed and assessed daily for clinical signs of EAE by two blinded independent observers during the acute phase of illness, and followed three times a week during the later chronic phase. The monophasic EAE MOG C57BL6 model was monitored for 28-30 days and the PLP SJL/J relapsing model was followed for nearly 6 months after immunization. The clinical scoring system used to quantify behavioral neurological deficits in the EAE mouse models (Table 2) was previously described (Aquino D, et al. Journal of Neurochemistry 1990:54:1398-404).

TABLE 2
Clinical scoring system for quantifying neurological deficit in
mouse EAE models.

| Score | Characteristics |
|---|---|
| 0 | Normal |
| 1 | Tail limb drop |
| 2 | Mild hind-leg weakness |
| 3 | Severe hind-leg weakness (paralysis) |
| 4 | Hind-leg paralysis plus mild front-limb involvement |
| 5 | Quadriplegic |
| 6 | Moribund or dead |

Bioluminescence Imaging (BLI). Bioluminescent signals were quantified using the In Vivo Imaging System 100 (IVIS: Xenogen, Alameda, CA) with a cold Charged Coupled Device (CCD) camera mounted in a dark box. Three animals were imaged simultaneously. Mice received an IV injection of 1 mg/kg D-luciferin (Xenogen) 2-3 minutes prior to imaging and were immediately anesthetized with vaporized isoflurane for imaging. The imaging signal was quantified in units of photons per second per centimeter squared per steradian (photons/s·cm2/sr) using LIVINGIMAGE Version 3.1 (Xenogen) software and integrated over 2 minutes. For signal quantification, photons were obtained from a “region of interest” (ROI) that covered 2.18 cm2 of contiguous forebrain and 5.27 cm2 of spinal cord region. Bioluminescence was expressed as a ratio of the total photons value from the central nervous system ROI to the photon value obtained from an equal sized area over the left ear, used as an endogenous control. In the acute EAE model, measurements were typically taken every 1-2 days and the average of 2 consecutive days readings were used. Bioluminescence imaging was performed every other day after disease onset and twice weekly during the later chronic phase of the illness.

JM4 treatment. The JM4 peptide was first dissolved in distilled water to 2 mg/ml and stored at −80° C. Immediately before use, the peptide solution was further diluted to 5 μg/200 μl with PBS. Treatment was initiated when the bioluminescence signal of either the brain or spinal cord became significantly higher than background (usually 8-9 days post-immunization and prior to onset of clinical signs). The EAE mice were randomly divided into a JM4 treated and a sham treated group. The JM4 treated EAE animals (4-10 per group) were treated daily with IV JM4 250 μg/kg of the peptide in PBS for 10-12 days. Control EAE animals were sham-treated with IV saline for the same time period. To evaluate the treatment effects, mice underwent BLI examination as well as clinical and histopathologic evaluation.

Real time PCR. To verify the correlation between GFAP message levels and Luciferase expression, total RNA from EAE mouse brain was extracted at different time points after immunization. A two-step real-time PCR was performed on an ABI 7700 Sequence Detection System (PE Applied Biosystems, Foster City, CA, USA) using the SYBR-Green I Master Kit (Roche Diagnostics, Indianapolis, IN, USA). cDNA was synthesized via RT-PCR (manufacturer's protocol) using SuperScript VILO (Invitrogen) on 2 μg of total RNA extracted with Trizol (Invitrogen). Two μL of 20 times-diluted RT-PCR reaction solution was tested in the real-time PCR reaction. The primer pair used to amplify the GFAP transcript was: forward 5″-ATGGTGATGCGGTTTTCTCTTC-3″ (SEQ ID NO: 16) and reverse 5′-CACGAACGAGTCCCTAGAGC-3′ (SEQ ID NO: 17), and for the luciferase transcript forward: 5′-GCTTTTGGCGAAGAATGAAA-3′ (SEQ ID NO: 18) and reverse 5′-CATTCCGCATACTGAGATTT-3′ (SEQ ID NO: 19). The real-time PCR was run for 40 cycles at: 94ºC for 25 seconds, 60ºC for 25 seconds, and 72ºC for 45 seconds. Quantification was performed using the relative standard curve method described in User Bulletin #2 for the ABI Prism 7700 from PE Applied Biosystems. The product of HPRTI (hypoxanthine-guanine phosphoribosyltransferase 1) transcript was used as an endogenous control. Standard curves were generated using 6 serial dilutions and a correlation score of >0).99 was observed for each run. Samples were run in triplicate and the average Ct value was used for analysis. The melting temperature was studied with a dissociation curve and PCR products were verified by electrophoresis in a 1% agarose gel.

Histopathology. To assess CNS inflammation and demyelination at different time points after immunization, EAE mice in the JM4 peptide-treated and sham-treated groups were anesthetized and perfused with ice-cold PBS, followed by 4% paraformaldehyde into the left ventricle. Spinal cords and brain were removed. Tissues were then embedded in paraffin, sectioned and stained with Luxol-Fast Blue/PAS to determine the extent of demyelination. Five-micron paraffin sections from the high cervical, thoracic and low lumbosacral region of the spinal cord were placed on the same slide. Each slide contained a control normal spinal cord section, a sham-treated EAE cord, and spinal cord sections from JM4 treated EAE animals. Histopathologic examination was performed in blinded fashion. In addition, SMI-32 anti-neurofilament H immunohistochemical staining was completed to assess acute axonal injury (Aquino D, et al. Journal of Neurochemistry 1990:54:1398-404). In brief, 5 μm sections were de-paraffinized, treated with antigen-retrieving solution, and blocked with normal horse serum to minimize nonspecific binding. The sections were first incubated with mouse SMI-32 antibody (Sternberger Monoclonals, Lutherville, MD) diluted 1:5,000 in PBS overnight at 4° C. After two 5-minute PBS washes, the sections were incubated with biotinylated horse anti-mouse IgG (Vector Labs, Burlingame, CA), and then by horseradish peroxidase (HRP)-avidin complex (Vector Labs) for 30 minutes each. Immunostaining was visualized by reacting the sections with diaminobenzidine tetrahydrochloride (Stable DAB: Invitrogen) for 1 to 3 minutes. Excess DAB was removed by washing with distilled water and the slides were evaluated using light microscopy.

Spinal cord (cut into 6-7 equal pieces from cervical to sacral) was frozen and serially cut at 8 μm-thick cross sections and collected on slides, then post-fixed in acetone at −20° C. for 10 min. Sections were first treated with Target Unmasking Fluid (Pan Path) at 90° C. for 10 min, and blocked in 2% normal horse serum for 10 min. Sections were incubated overnight at 4° C. with either monoclonal rat anti-mouse C3 complement (Abcam, AB11862) at 1:50 or polyclonal rabbit anti-mouse GFAP (Dako Z0334GFAP) at 1:500. The next day sections were washed in PBS three times for 10 min each and incubated with cy3 or cy2 conjugated goat anti-mouse/rat/rabbit IgG (Jackson ImmunResearch) at 1:120 working dilution for 1 hour. Sections were washed in dH2O three times for 10 min each, followed by fluorescence microscopy imaging.

Immunohistochemistry images were obtained using an Olympus BX41 fluorescent microscope (Center Valley, PA, USA) fitted with ProgRes MF cool digital camera using 4× or 10× objectives. Half of each section from different spinal levels was quantified by measuring the percent area in the structure where label exceeded a constant threshold value using Image Pro 4.0 image analysis software (BD Biosciences, Franklin Lakes, NJ).

Statistical analysis. Data is presented as the mean±SEM. Two tailed t-tests were used to compare JM4 effects on BLI and clinical scores. The long-term effects of JM4 were studied in the relapsing EAE model by using each subject's average clinical score from day 60 onward. Bioluminescence was measured by the number of days where luminescence intensity in the spinal cord exceeded a threshold of 0.5 relative to the left ear. Survival data was examined using a two-tailed log-rank test. Values of p<0.05 were considered significant.

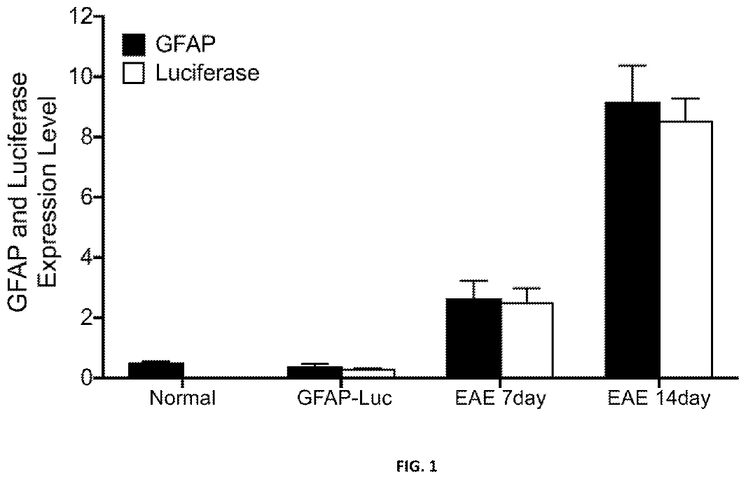

Results. Verification of correlation between GFAP and luciferase expression. Quantitative real time PCR was used to verify the correlation between GFAP expression and luciferase expression in the GFAP-Luc/SJL EAE mouse model. RNA was extracted from SJL/J EAE mice brains at 0, 7, and 14 days post immunization and two-step real-time PCR was performed. Increased expression of GFAP and luciferase was seen at days 7 and 14 (FIG. 1). A strong correlation (>98%) was noted between GFAP and luciferase transcription (r square=0.9958).

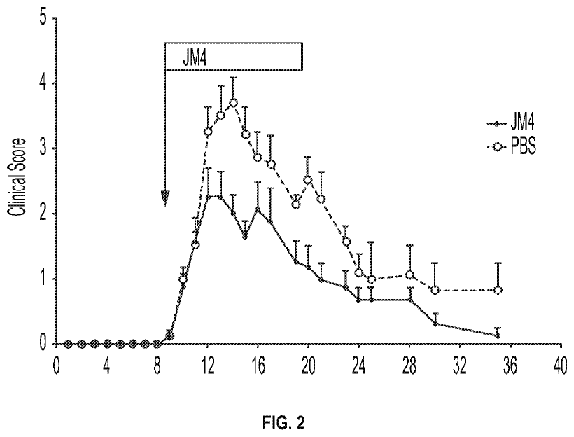

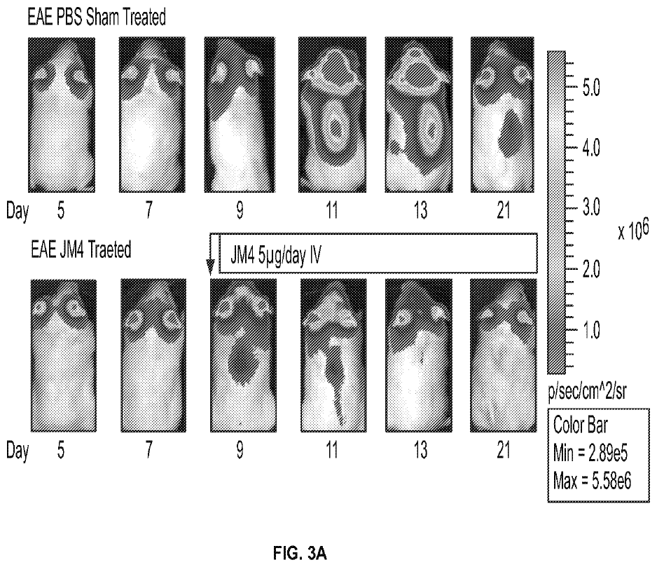

Effect of JM4 treatment on bioluminescence in monophasic and relapsing-remitting EAE models. Luo et al. (Luo J, et al. Journal of Neuroinflammation 2008:5:6) used GFAP-Luc C57BL/6j-Tyrc-2j albino mice to study MOG-induced acute EAE and found a close correlation between GFAP bioluminescence and clinical scores were used. The GFAP-Luc model was used herein to investigate the therapeutic effects of JM4 on MOG induced monophasic EAE. Female FI offspring of FVB/N-Tg (GFAP-luc+/−) crossed with C57BL/6j-Tyre-2j were immunized with 200 μg MOG. These mice later received either JM4 250 μg/kg IV or sham treatment with 0.9% saline IV for 12 days starting on day 9 post-immunization. Disease onset and severity were assessed by clinical neurologic scoring as well as by GFAP-luc BLI. A monophasic clinical course was observed as described by Luo et al. (Wang B, et al. Neurotherapeutics 2016:2:418-427) in the sham treated EAE group. Mice first developed clinical signs on day 10±0.7 and reached a maximum mean clinical score of 3.7±0.4 on day 14, with approximate disease duration of two weeks. In contrast. EAE animals treated with JM4 showed a significant reduction in clinical score from day 12 throughout the remainder of the disease course (FIG. 2), and the maximum mean clinical score was reduced to 2.25±0.3 (p<0.05).

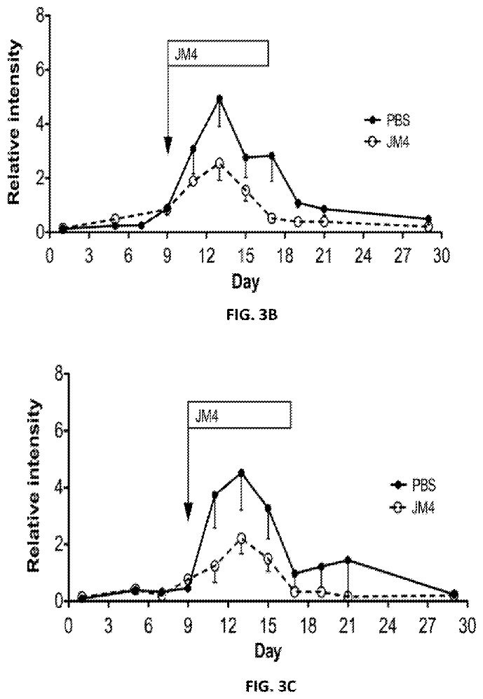

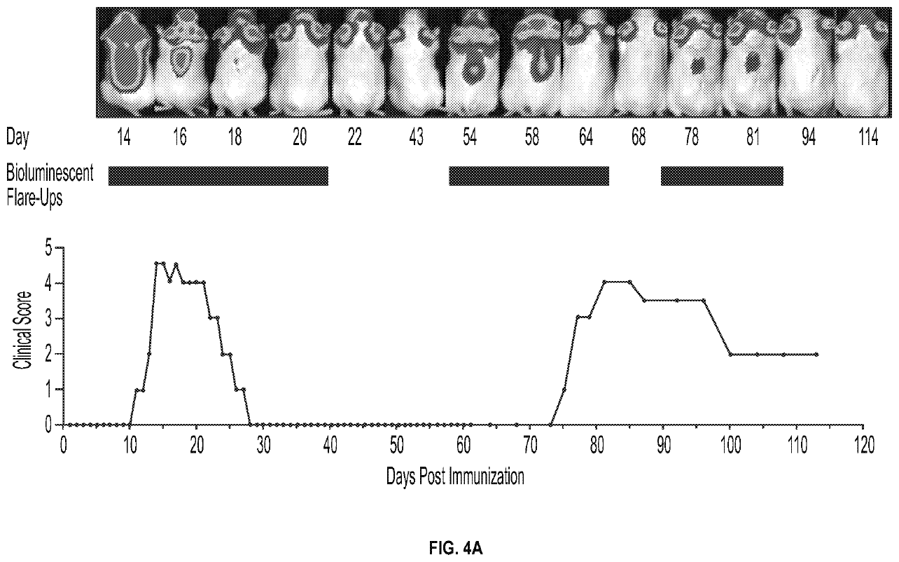

A marked positive treatment effect with JM4 was also seen by GFAP-luc BLI assessment that correlated with clinical scores (FIG. 3). In the MOG-induced EAE model, earliest detectable bioluminescent signal (ROI/Ear ratio ≥0.4) was noted over the forebrain as early as 7 days post-immunization. A significant signal was detected in 90% of animals by 9 days, maximum values were observed between days 11 to 16, typically preceding onset of clinical deficit by 2-3 days. Animals with similar bioluminescent scores at day 9 were paired into JM4-treated or sham-treated EAE groups and J M4 treatment was initiated. Peak bioluminescent readings within the brain and spinal cord regions over the disease course in the JM4 treated MOG EAE group remained significantly lower than those of the sham-treated group (FIG. 3). A 3-4-fold reduction from peak disease scores (days 11-17) at days 11 to 12 was seen in JM4 treated compared with sham animals. In addition. GFAP-luc signal returned to baseline 3-4 days earlier in JM4 treated animals when compared to the sham-treated group.

Sustained effect of JM4 treatment on the PLP induced relapsing-remitting EAE model. After quantifying the short-term beneficial effects of JM4 peptide in the acute monophasic MOG-induced EAE mouse model, the long term JM4 treatment effects on chronic relapsing-remitting PLP antigen-induced SJL/J EAE mice was investigated. It was tested whether monitoring GFAP-luc expression with bioluminescent imaging might be a more sensitive and comprehensive indicator for determining CNS neuroinflammatory status than clinical examination. For this, a new light-producing GFAP-Luc/SJL/1 EAE model was created by crossing GFAP-Luc (−/+)/FVBN (Xenogen) mice with SJL/J mice.

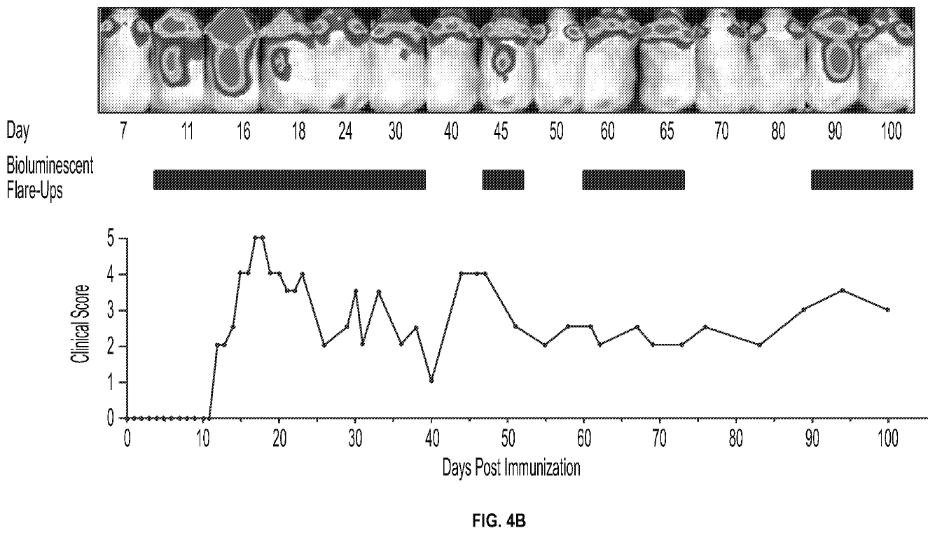

The FI offspring SJL/J female mice were initially immunized with 100 μg of PLP139-151 and a booster immunization was administered on Day 7. The mice were evaluated by clinical exam and BLI imaging for up to 6 months. Eighty percent of PLP immunized mice developed EAE and showed at least one relapse as assessed by clinical scoring during the 5-6-month experimental course. The first clinical signs appeared 11±1.5 days post-immunization, with clinical scores reaching a peak at 14±2.4 days and on average the acute peak lasted 7.5±3.8 days. Bioluminescent signal above background level was detected at 9 days in 80% of animals. Increased signals were first seen in the forebrain and subsequently extended to spinal cord in 1-3 days with spinal cord signals typically peaking 1-2 days prior to the onset of clinical manifestations. The intensity of photon readings in the spinal cord correlated strongly with the clinical score. In twenty percent of PLP immunized mice, no clinical symptoms or increased bioluminescent signal in the brain was seen; these mice were removed from the study. Prior to a definite clinical recurrence, the animals exhibited a substantial increase in bioluminescent signal in the spinal cord that subsequently receded to background levels over the course of a few days. Bioluminescent intensity increases in the rostral forebrain were not always followed by clinical evidence of disease (FIG. 4).

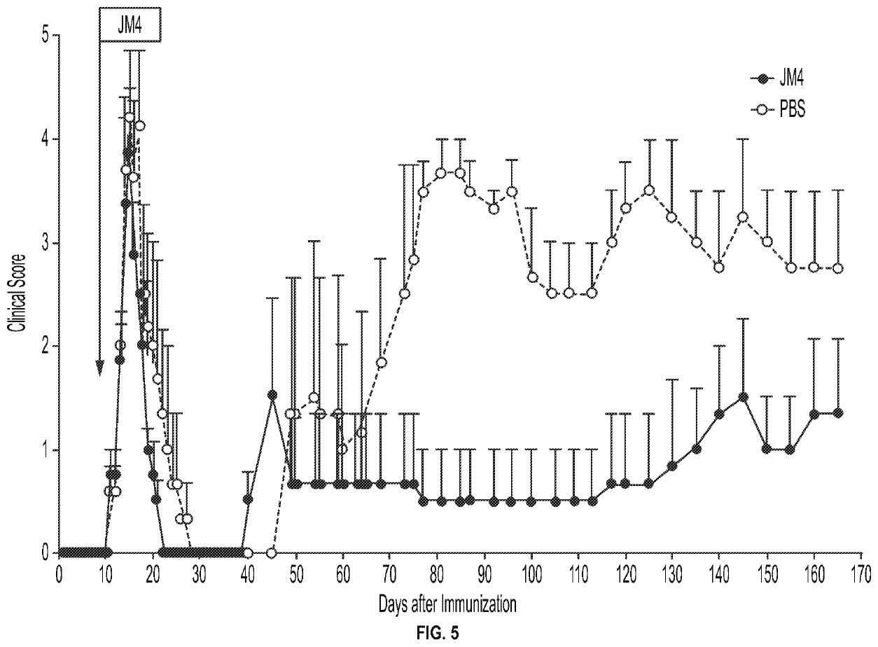

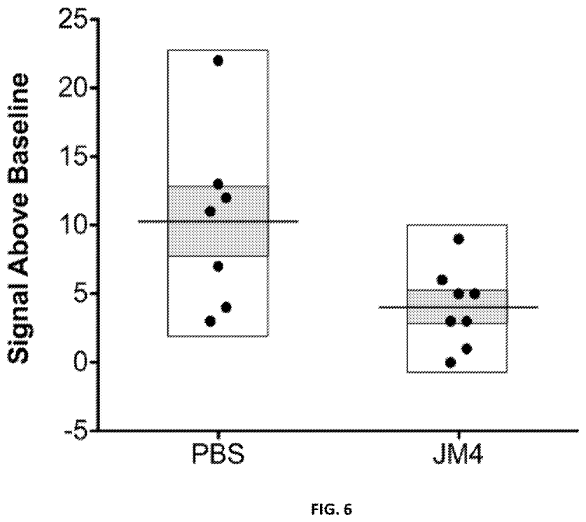

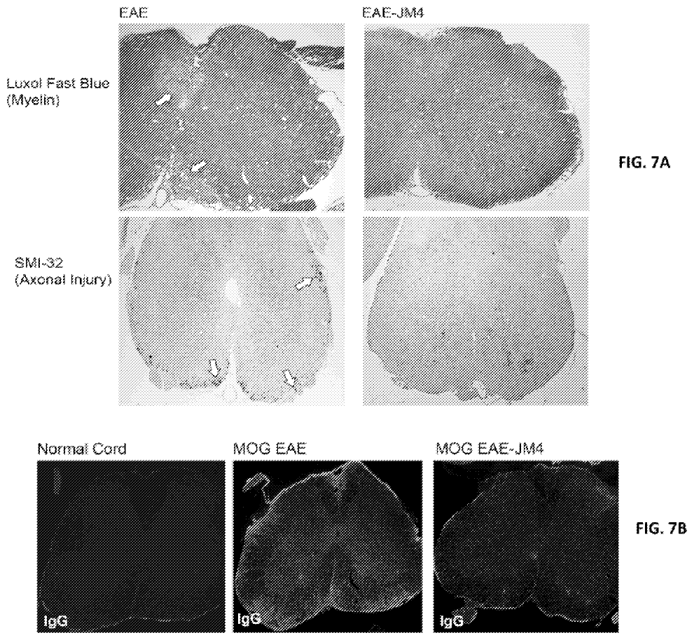

After characterizing the new light-producing SJL/J EAE mouse model on day 9, the mice (4-6 animals in each experiment) were divided into two groups, one treated with a 12-day course of IV JM4 peptide (5 μg/day) and the other sham-treated (0.9% saline IV). Clinical deficits were scored daily, and images were taken 2-3 times weekly during the acute stage and followed by imaging every 7-10 days for up to 5 months. Long term clinical scores of JM4 treated relapsing-remitting mice remained significantly lower than sham treated EAE mice for over five months (FIG. 5). Clinical deficit in JM4 treated animals rapidly improved 7 days earlier than sham-treated animals during the acute phase of the disease. The average clinical score for JM4 treated mice remained under 1 for 130 days, while sham-treated group scores centered around 2 for the same period. In JM4 treated relapsing-remitting EAE mice, both GFAP-luc expression and total number of disease flare-ups in either the brain or spinal cord were significantly decreased compared to sham treated animals (FIG. 6). SJL/J relapsing-remitting EAE mice treated with JM4 showed a large reduction in number of injured axons rimming the ventral spinal cord compared to sham treated control mice (FIG. 7). Treatment with JM4 led to 100% survival in relapsing-remitting SJL/J mice at 100 days, whereas 50% of sham treated animals survived, as analyzed by Kaplan-Meier Plot.

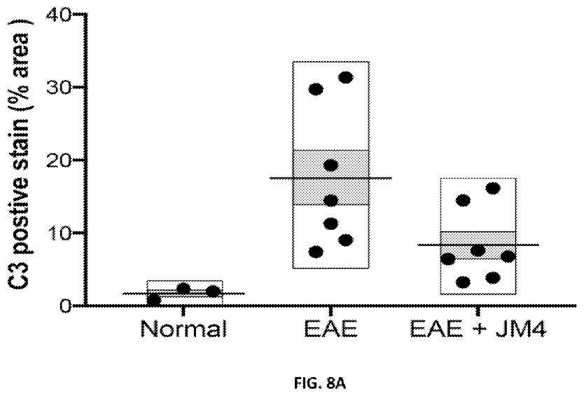

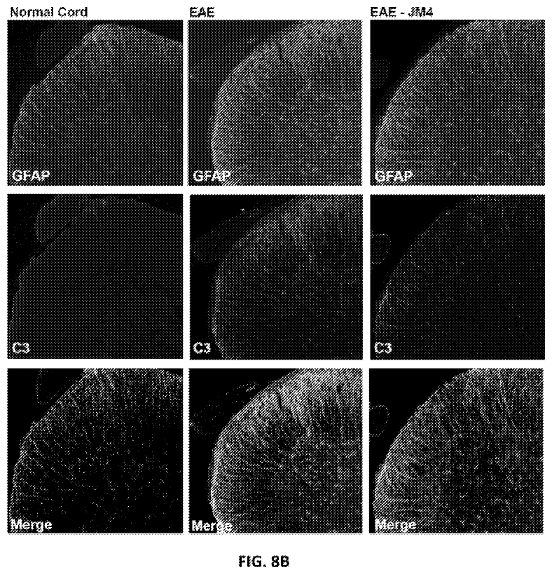

A1 Astrocyte activation represented by (3 expression in MOG-induced monophasic EAE mice was attenuated by JM-4 treatment. MOG-induced EAE mice received either JM4 250 μg/kg IV or sham treatment with 0.9% saline IV for 12 days starting on day 9 post-immunization. To show A1 astrocyte activation in the CNS of EAE mice, immunohistochemistry for complement component C3, a marker for A1 astrocytes, was performed. C3 expression colocalizing with GFAP was upregulated in the spinal cord of the C57BL MOG EAE mouse 24 days post immunization compared to sham-treated control mice. Importantly, treatment with JM4 led to a significant reduction in C3 expression (n=7 per group, EAE 17.5+/−3.7 versus JM4 treated 8.3+/−1.9, p<0.05, ANOVA followed by Bonferroni comparison). These results show that JM-4 treatment attenuated A1 astrocyte activation by over 50% in EAE mice. See, for example, FIG. 8.

Discussion. The BLI/clinical study demonstrates the profound immunomodulatory benefit of the small EPO derived peptide, JM4, in both the acute MOG and relapsing-remitting PLP SJL/J EAE mouse models. In both models, the positive clinical and imaging treatment effects of JM-4 were sustained without the negative hematogenic side effects associated with whole molecule EPO.

Traditional treatments for RRMS, including oral and injectable disease modifying therapies, have usually demonstrated beneficial effects that are limited to the duration of therapy. An emerging class of treatments for MS called immune reconstitution therapies show markedly prolonged clinical benefits that last well beyond the brief treatment course. Two immune reconstitution compounds have been recognized: alemtuzumab and cladribine; these compounds deplete lymphocytes, class switched and unswitched memory B cells, and lead to a reduction in relapse rate and gadolinium enhancing lesions as seen on brain magnetic resonance imaging (MRI) in MS patients. With both medications, the prolonged clinical benefit of therapy is often retained for months to years after the time of active treatment (Sorensen P and Sellebjerg F. Therapeutic Advances in Neurological Disorders 2019:12:1-16). These findings that short-term therapy with JM-4 leads to sustained clinical benefits and downregulation of astrocyte activation as monitored by BLI for at least five months, point to JM-4 as a therapy for achieving long-lasting benefits akin to the immune reconstitution therapies.

In the experiments described herein, relative GFAP-luc expression was noted to increase over time in the EAE transgenic mouse model, reinforcing the previously recognized concept of GFAP, the classic astrocyte marker, as a useful biomarker of neuro-inflammation (Luo J, et al. Journal of Neuroinflammation 2008:5:6). These outcomes were similar to previously studied acute EAE models showing increased GFAP mRNA levels coinciding with clinical symptoms and inflammation (Smith M, et al. Brain Research 1983:264:241-53; Aquino D, et al. Journal of Neurochemistry 1990:54:1398-404; and Aquinno D, et al. Journal of Neurochemistry 1988:51:1085-1096). In the relapsing-remitting SJL/J EAE mouse model, enhanced bioluminescent abnormalities were observed more frequently than clinical deficits, leading to noninvasively monitor the effect of treatment at multiple time points and with greater accuracy prior to the appearance of clinical symptoms. These findings suggest that BLI is often more sensitive than clinical assessment in mice. Similarly, prior work using BLI reported early detection of EAE lesions 5-7 days before the development of clinical neurologic impairment (Luo J, et al. Journal of Neuroinflammation 2008:5:6).

Analogous to the use of BLI for monitoring treatment response in EAE mouse models, MRI has been utilized for diagnostic purposes and to monitor disease response to therapy in MS patients. Similar to the findings of this current study, acute MS lesions seen on MRI may often precede clinical signs and symptoms (Barkhof F, et al. Brain 1997:120:2059-69; and Lincoln J, et al. Archives of Neurology 2009:66:412-4). Interestingly, the results described herein showed that bioluminescent intensity increases in the rostral brain were not always followed by clinical evidence of disease. These findings are reminiscent of the well-recognized clinico-radiogical paradox in MS, where radiographic lesion load seen on MRI often does not correlate with the extent of clinical disability (Barkhof F. Current Opinion in Neurology 2002:15:239-245; and Wuerfel J, et al. European Journal of Neuroscience 2007:26:190-198).

As anticipated. BLI appeared to be of greater utility in the assessment of disease status in the chronic remitting-relapsing EAE model. Although the average daily intensity of the bioluminescent signal was not significantly different between the JM4 and sham-treated groups, the number of flareups in JM-4 treated animals where GFAP expression was above background was always substantially less than that seen with sham-treated animals (FIG. 6). This data shows that a 10-12 day course of treatment with JM-4 halts the onset of severe EAE in the acute phase and markedly reduces the number of inflammatory recurrences in chronic EAE animals for more than 5 months. By clinical scoring, 50% of JM4 treated SJL/J animals remained relapse-free throughout the 5-month experiment, whereas 20% of sham-treated mice remained relapse-free. Notably, JM-4 therapy for 12 days resulted in 100% survival of SJL/J EAE animals, whereas half of the sham treated SJL/J EAE animals died.

The presence of the toxic A1 astrocytic phenotype was detected in the spinal cord of EAE mice, as revealed by a strong upregulation of C3 protein on GFAP positive astrocytes. This result is consistent with the reported presence of A1 reactive astrocytes in MS and other neurodegenerative disorders (Ponath G, et al. Frontiers in Immunology 2018:9:1-12; Colombo E and Farina C. Trends in Immunology 2016:37:608-620; and Liddelow S and Barres B. Immunity 2017:46:957-966). Most importantly, JM-4 treatment of EAE mice lead to massive reduction of C3 positive A1 astrocytes as detected by immunohistochemistry as well as reduction of GFAP-luc expression detected by BLI. These results suggest profound effects on the immune system in the CNS by JM-4 treatment in addition to its effect on the peripheral immune system including changes in subsets of T cells and proinflammatory cytokines as we previously reported (Yuan R, et al. Neurotherapeutics 2015:12:850-861). Further characterization of astrocyte types in the brain of EAE mouse models are needed for future development of effective drugs to treat EAE.

As described herein, it was shown that a short-term treatment pulse with JM-4 lasting 7-14 days led to sustained and long-term clinical benefit lasting at least five months in the chronic relapsing EAE mouse. Thus, these data suggest that a short course of JM-4 will lead to prolonged clinical benefits without side effects in the treatment of MS patients.

What is claimed is:

1. A dosing regimen comprising at least one treatment cycle followed by a rest phase, wherein the treatment cycle comprises administering an effective amount of an erythropoietin (EPO)-derived peptide to allow for a sustained therapeutic effect after withdrawal of the EPO-derived peptide, wherein the EPO-derived peptide is not administered during the rest phase.

2. The dosing regimen of claim 1, wherein the treatment cycle comprises administering an effective amount of the EPO-derived peptide daily for 7-14 days.

3. The dosing regimen of claim 1, wherein the treatment cycle further comprises a second treatment cycle after the rest phase.

4. A method of treating relapsing-remitting multiple sclerosis, the method comprising administering to a subject an effective amount of an erythropoietin (EPO)-derived peptide for at least one treatment cycle, wherein the treatment cycle comprises an effective amount of the EPO-derived peptide to allow for a sustained therapeutic effect after withdrawal of the EPO-derived peptide, wherein the treatment cycle is followed by a rest phase, and wherein the EPO-derived peptide is not administered during the rest phase.

5. The method of claim 4, wherein the rest phase is at least 5 months.

6. The method of claim 4, further comprising a second treatment cycle after the rest phase.

7. The method of claim 6, wherein the second treatment cycle is administered after the rest phase.

8. The method of claim 6, wherein the second treatment cycle is administered one year from the beginning of the initial treatment cycle.

9. The method of claim 4, further comprising administering a therapeutic other than the EPO-derived peptide during the rest phase.

10. The method of any of the preceding claims, wherein the EPO-derived peptide is GCAEHCSLNENITVPDTKV (SEQ ID NO: 1).

11. The dosing regimen of claim 10, wherein the EPO-derived peptide is end protected with an acetyl group protecting the amino terminus and an amide group protecting the carboxyl terminus.

12. A dosing regimen comprising at least one treatment cycle followed by a rest phase, wherein the treatment cycle comprises administering an effective amount of an erythropoietin (EPO)-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the EPO-derived peptide is not administered during the rest phase.

13. The dosing regimen of claim 12, wherein the EPO-derived peptide is end protected with an acetyl group protecting the amino terminus and an amide group protecting the carboxyl terminus.

14. A method of treating relapsing-remitting multiple sclerosis, the method comprising administering to a subject an effective amount of an erythropoietin (EPO)-derived peptide for at least one treatment cycle, wherein the treatment cycle comprises administering an effective amount of the EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the treatment cycle is followed by a rest phase, wherein the EPO-derived peptide is not administered during the rest phase.

15. A method of reducing A1 astrocyte activation in spinal cord, the method comprising administering to a subject an effective amount of an erythropoietin (EPO)-derived peptide for at least one treatment cycle, wherein the treatment cycle comprises administering an effective amount of the EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the treatment cycle is followed by a rest phase, wherein the EPO-derived peptide is not administered during the rest phase.

16. A method of decreasing complement component C3, the method comprising administering to a subject an effective amount of an erythropoietin (EPO)-derived peptide for at least one treatment cycle, wherein the treatment cycle comprises administering an effective amount of the EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the treatment cycle is followed by a rest phase, wherein the EPO-derived peptide is not administered during the rest phase.

17. A method of treating a disease, disorder or condition having an inflammatory or autoimmune component in a subject in need thereof, the method comprising administering to a subject an effective amount of an erythropoietin (EPO)-derived peptide for at least one treatment cycle, wherein the treatment cycle comprises administering an effective amount of the EPO-derived peptide to allow for a sustained therapeutic effect after withdrawal of the EPO-derived peptide, wherein the EPO-derived peptide consists of the amino acid sequence GCAEHCSLNENITVPDTKV (SEQ ID NO: 1), wherein the treatment cycle is followed by a rest phase, wherein the EPO-derived peptide is not administered during the rest phase, wherein the composition is effective at ameliorating at least one symptom from at least one disease, disorder, or condition having an inflammatory or autoimmune component.

18. The method of claim 17, wherein the disease, disorder or condition having an inflammatory or autoimmune component is dementia, acute cerebrovascular injury, acute spinal cord injury, acute traumatic brain injury and repetitive mild traumatic brain injury, acute cardiovascular injury, arthritis, autoimmune disease, demyelinating disease, a stroke, multiple sclerosis, a neurological injury and immune-mediated inflammation.

19. Use of an erythropoietin (EPO)-derived peptide for the production of a medicament for the treatment of multiple sclerosis (MS) in a patient, the treatment comprising a first treatment cycle of the EPO-derived peptide followed by at least one further treatment cycle of the EPO-derived peptide, in which each treatment cycle comprises 1-14 doses which are applied on consecutive days, wherein the daily dose is >0 and ≤ 10 mg, and wherein each treatment cycle is separated from the next treatment cycle by at least 1-24 months.

20. The use of claim 19, wherein the at least one further treatment cycle is to be administered at least 5 months after the first treatment cycle.

21. The use of claim 19 or claim 20, wherein the at least one further treatment cycle is at the same daily dose for a shorter duration than the first treatment cycle.

22. The use of any one of claims 19 to 21, wherein the first treatment cycle of the EPO-derived peptide is at a dose of 5-10 mg/day for five days.

23. The use of claim 22, wherein the patient is to be retreated at 12 months after the first treatment cycle with a further treatment cycle of the EPO-derived peptide at a dose of 5 μg/day for 1 week.

24. The use of any preceding claims, wherein two initial treatment cycles of the EPO-derived peptide are to be followed by a third or subsequent treatment cycle of the EPO-derived peptide only upon evidence of renewed MS activity.

25. The use of claim 24, wherein the third or subsequent treatment cycle is at a dose of 5-10 mg/day for 1 week.

26. The use of claim 24 or claim 25, wherein evidence of renewed MS activity is diagnosed by clinical means.

27. The use of claim 26, wherein the clinical means are selected from the group consisting of relapse or progression of neurological disability.

28. The use of claim 24 or claim 25, wherein evidence of renewed MS activity is diagnosed by magnetic resonance imaging (MRI) of the brain or spinal cord.

29. The use of claim 29, wherein MS activity detected by MRI is indicated by the occurrence of new cerebral or spinal lesions on T1 or T2 weighted images or by the increase in volume of such lesions.

30. The use of claim 29 or claim 31, wherein repeated MRIs are performed at fixed intervals after the second treatment cycle of the EPO-derived peptide in order to determine whether a third or subsequent treatment cycle of the EPO-derived peptide is necessary.

31. The use of claim 30, wherein the third or subsequent treatment cycle of the EPO-derived peptide is performed before the disease re-manifests clinically.

32. The use according to any preceding claim, wherein the EPO-derived peptide is to be administered intravenously.

33. The use according to any preceding claim, wherein the MS is relapsing MS.

34. The use according to any preceding claim, wherein the patient has received prior therapy for MS.

35. The use according to any of the preceding claims, wherein the EPO-derived peptide consists of the amino acid sequence GCAEHCSLNENITVPDTKV (SEQ ID NO: 1).


SEQUENCE LISTING (19 sequences)

SEQ ID NO: 1 - protein, 19 aa, Artificial Sequence
  Synthetic construct
GCAEHCSLNENITVPDTKV

SEQ ID NO: 2 - protein, 14 aa, Artificial Sequence
  Synthetic construct
CAEHCSLNENITVP

SEQ ID NO: 3 - protein, 17 aa, Artificial Sequence
  Synthetic construct
TTGCAEHCSLNENITVP

SEQ ID NO: 4 - protein, 18 aa, Artificial Sequence
  Synthetic construct
CAEHCSLNENITVPDTKV

SEQ ID NO: 5 - protein, 21 aa, Artificial Sequence
  Synthetic construct
TTGCAEHCSLNENITVPDTKV

SEQ ID NO: 6 - protein, 17 aa, Artificial Sequence
  Synthetic construct
TTGCAEHCSLNENITVP

SEQ ID NO: 7 - protein, 25 aa, Artificial Sequence
  Synthetic construct
CAEHCSLKHQGLNKNINLDSVDGVP

SEQ ID NO: 8 - protein, 18 aa, Artificial Sequence
  Synthetic construct
GCAEHCSLMENNLRRPNL

SEQ ID NO: 9 - protein, 16 aa, Artificial Sequence
  Synthetic construct
AEHCSLMENNLRRPNL

SEQ ID NO: 10 - protein, 193 aa, Artificial Sequence
  Synthetic construct
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 11 - protein, 27 aa, Artificial Sequence
  Synthetic construct
MGVHECPAWLWLLLSLLSLPLGLPVLG

SEQ ID NO: 12 - protein, 166 aa, Artificial Sequence
  Synthetic construct
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 13 - protein, 4 aa, Artificial Sequence
  Synthetic construct
TGDR

SEQ ID NO: 14 - protein, 165 aa, Artificial Sequence
  Synthetic construct
APPRLICDSRVLERYLLEAKEAENITTGCAEHCSLNENITVPNTKVNFYAWKRMEVGQQAVEVWQGLALLSEAVLRGQALLVNSSQPWEPLQLHVDLAVSGLRELTTLLRALGAQLEAISPPDAASAAPLATITANTERKLERVYSNALRGKLKLYTQEACRTGD

SEQ ID NO: 15 - protein, 13 aa, Artificial Sequence
  Synthetic construct
HSLGKWLGHPDKF

SEQ ID NO: 16 - DNA, 22 bp, Artificial Sequence
  Synthetic construct
atggtgatgcggttttctcttc

SEQ ID NO: 17 - DNA, 20 bp, Artificial Sequence
  Synthetic construct
cacgaacgagtccctagagc

SEQ ID NO: 18 - DNA, 20 bp, Artificial Sequence
  Synthetic construct
gcttttggcgaagaatgaaa

SEQ ID NO: 19 - DNA, 20 bp, Artificial Sequence
  Synthetic construct
cattccgcatactgagattt